(19) United States
(12) Patent Application Publication
(10) Pub. No.: US 2025/0025520 A1
(43) Pub. Date: Jan. 23, 2025

(54) MICROBE-BASED MODULATION OF SEROTONIN BIOSYNTHESIS

(71) Applicant: The Regents of the University of California, Oakland, CA (US)

(72) Inventors: Elaine Hsiao, Rowland Heights, CA (US); Jessica Yano, Pasadena, CA (US)

(21) Appl. No.: 18/416,203

(22) Filed: Jan. 18, 2024

Related U.S. Application Data
(63) Continuation of application No. 18/318,620, filed on May 16, 2023, now abandoned.
Application No. 18/318,620 is a continuation of application No. 14/839,041, filed on Aug. 28, 2015, now abandoned.
(60) Provisional application No. 62/147,741, filed on Apr. 15, 2015.
(60) Provisional application No. 62/090,818, filed on Dec. 11, 2014.
(60) Provisional application No. 62/045,467, filed on Sep. 3, 2014.

(51) Int. Cl. A61K 35/747; A61K 35/742

(52) U.S. Cl. CPC A61K 35/747; A61K 35/742

(57) ABSTRACT

Methods and compositions that can be used to modulate serotonin level in a subject are disclosed herein. In some embodiments, the methods include adjusting the composition of gut microbiota in the subject. Also disclosed are methods of adjusting the level of one or more serotonin-related metabolites to modulate serotonin biosynthesis in a subject, and methods for treating serotonin-related diseases, for example disorders caused by serotonin deficiency.

### RELATED APPLICATIONS

The present application is a continuation of U.S. application Ser. No. 18/318,620, filed May 16, 2023, which is a continuation of U.S. application Ser. No. 14/839,041, filed Aug. 28, 2015, which claims priority under 35 U.S.C. § 119 (e) to U.S. Provisional Application No. 62/045,467, filed on Sep. 3, 2014; U.S. Provisional Application No. 62/090,818, filed on Dec. 11, 2014; and U.S. Provisional Application No. 62/147,741, filed on Apr. 15, 2015. The content of these priority applications is herein expressly incorporated by reference in their entirety.

### STATEMENT REGARDING FEDERALLY SPONSORED R&D

This invention was made with government support under grant no. 5DP5OD017924 awarded by the National Institutes of Health. The government has certain rights in the invention.

### BACKGROUND

### Field

The present disclosure relates generally to the field of modulation of serotonin biosynthesis and treatment of serotonin-related diseases.

### Description of the Related Art

Serotonin (5-hydroxytryptamine (5-HT)) is a monoamine neurotransmitter. Biochemically derived from tryptophan, serotonin is primarily found in the gastrointestinal tract (GI tract), blood platelets, and the central nervous system (CNS) of humans. Serotonin regulates a variety of biological processes, for example intestinal movements, platelet activation/aggregation, stimulation of myenteric neurons and gut mobility, mood, appetite, sleep, some cognitive functions such as memory and learning, bone metabolism, and cardiac functions. Abnormal level of serotonin in animals can cause pathological conditions including depression, anxiety, obsessive-compulsive disorder, irritable bowel syndrome, cardiovascular disease, osteoporosis, abnormal gastrointestinal motility, abnormal platelet aggregation, abnormal platelet activation, and abnormal immune response.

Serotonin deficiency thus presents a health risk. Various drugs have been developed to treat serotonin deficiency, such as selective serotonin reuptake inhibitors (SSRI drugs) and monoamine oxidase inhibitors (MAO inhibitors). However, there is a need to provide effective treatment with no or little side effects to serotonin-related diseases.

### SUMMARY

The present disclosure provides a method for modulating the level of serotonin in a subject. The method includes, in some embodiments, adjusting the composition of gut microbiota in a subject, and thereby changing the level of serotonin in the subject.

In some embodiments, the method further includes determining the level of serotonin in the subject before the composition of gut microbiota in the subject is adjusted, after the composition of gut microbiota in the subject is adjusted, or both. In some embodiments, the subject suffers from or is at a risk of developing a serotonin-related disease. In some embodiments, the serotonin-related disease is irritable bowel syndrome, inflammatory bowel disease, cardiovascular disease, osteoporosis, abnormal gastrointestinal motility, abnormal platelet aggregation, abnormal platelet activation, abnormal immune response, depression, anxiety, or a combination thereof. In some embodiments, the subject suffers from an abnormality in enteric motor and secretory reflexes, an abnormality in platelet aggregation, an abnormality in immune responses, an abnormality in bone development, an abnormality in cardiac function, an abnormality in gastrointestinal motility, an abnormality in hemostasis, an abnormality in mood, an abnormality in cognition, an abnormality in osteoblast differentiation, an abnormality in hepatic regeneration, an abnormality in erythropoiesis, an abnormality in intestinal immunity, an abnormality in neurodevelopment, or any combination thereof.

In some embodiments, adjusting the composition of gut microbiota increases expression of TPH1 gene in the subject. In some embodiments, adjusting the composition of gut microbiota increases serotonin biosynthesis from intestinal enterochromaffin cells in the subject. In some embodiments, changing the level of serotonin in the subject comprises changing one or more of the gut level, the colonic level, the peripheral level, the serum level, the plasma level, and the fecal level of serotonin in the subject. In some embodiments, adjusting the composition of gut microbiota enhances one or more of gastrointestinal motility, platelet activation, and platelet aggregation of the subject. In some embodiments, adjusting the composition of gut microbiota of the subject comprises fecal transplantation, microbiota conventionalization, microbial colonization, reconstitution of gut microbiota, probiotic treatment, antibiotic treatment, or a combination thereof.

In some embodiments, adjusting the composition of gut microbiota of the subject comprises administering to the subject a composition comprising one or more types of spore-forming bacteria. In some embodiments, the one or more types of spore-forming bacteria comprise Lactobacillales, Proteobacteria, Clostridia, or a mixture thereof. In some embodiments, the one or more types of spore-forming bacteria comprise Clostridia Cluster IV bacteria, Clostridia Cluster XIVa bacteria, or both. In some embodiments, the composition comprising one or more types of spore-forming bacteria comprises spore-forming microbes from a human intestine. In some embodiments, the composition comprising one or more types of spore-forming bacteria comprises spore-forming microbes from a healthy human colon. In some embodiments, at least 50% of the bacteria in the composition comprising one or more types of spore-forming bacteria are Clostridial species. In some embodiments, the composition comprising one or more types of spore-forming bacteria is a probiotic composition, a neutraceutical, a pharmaceutical composition, or a mixture thereof.

The present disclosure also provides a method for modulating serotonin biosynthesis in a subject. The method include, in some embodiments, determining the level of one or more serotonin-related metabolites in a subject; and adjusting the level of at least one of the one or more serotonin-related metabolites in the subject, and thereby modulating serotonin biosynthesis in the subject. In some embodiments, adjusting the level of at least one of the one or more serotonin-related metabolites comprises adjusting the composition of gut microbiota in the subject.

In some embodiments, adjusting the composition of gut microbiota of the subject comprises administering the subject a composition comprising one or more types of spore-forming bacteria. In some embodiments, the one or more types of spore-forming bacteria comprise Lactobacillales, Proteobacteria, Clostridia, or a mixture thereof. In some embodiments, the one or more types of spore-forming bacteria comprise Clostridia Cluster IV bacteria, Clostridia Cluster XIVa bacteria, or both. In some embodiments, the composition comprising one or more types of spore-forming bacteria comprises spore-forming microbes from a human intestine. In some embodiments, at least 50% of the bacteria in the composition comprising one or more types of spore-forming bacteria are Clostridial species. In some embodiments, the composition comprising one or more types of spore-forming bacteria is a probiotic composition, a neutraceutical, a pharmaceutical composition, or a mixture thereof.

In some embodiments, the one or more serotonin-related metabolites comprise at least one of the metabolites listed in Table 1. In some embodiments, adjusting the level of the serotonin-related metabolite in the subject comprises administering the serotonin-related metabolite to the subject. In some embodiments, adjusting the level of the serotonin-related metabolite in the subject comprises activating an enzyme involved in the in vivo synthesis of the serotonin-related metabolite, administering a substrate or an intermediate in the in vivo synthesis of the serotonin-related metabolite, or both. In some embodiments, adjusting the level of the serotonin-related metabolite in the subject comprises inhibiting an enzyme involved in the in vivo synthesis of the serotonin-related metabolite, administering to the subject an antibody against the serotonin-related metabolite, administering to the subject an antibody against an intermediate for the in vivo synthesis of the serotonin-related metabolite, administering to the subject an antibody against a substrate for the in vivo synthesis of the serotonin-related metabolite, or a combination thereof.

In some embodiments, the serotonin-related metabolite is deoxycholate, α-tocopherol, paminobenzoate, or tyramine. In some embodiments, adjusting the level of the serotonin-related metabolite improves gastrointestinal motility of the subject. In some embodiments, the method further includes determining the serotonin level of the subject after adjusting the level of the serotonin-related metabolite in the subject.

Also disclosed herein is a method for treating a disorder caused by serotonin deficiency. The method includes, in some embodiments, adjusting the composition of gut microbiota in a subject suffering from a disorder caused by serotonin deficiency; and increasing the colonic or blood level of serotonin in the subject.

In some embodiments, the method further includes determining the colonic or blood level of serotonin in the subject before the composition of gut microbiota in the subject is adjusted, after the composition of gut microbiota in the subject is adjusted, or both. In some embodiments, the disorder is irritable bowel syndrome, inflammatory bowel disease, cardiovascular disease, osteoporosis, abnormal gastrointestinal motility, abnormal platelet aggregation, abnormal platelet activation, abnormal immune response, depression, anxiety, or a combination thereof.

In some embodiments, adjusting the composition of gut microbiota of the subject comprises administering to the subject a composition comprising one or more types of spore-forming bacteria. In some embodiments, the one or more types of spore-forming bacteria comprise Lactobacillales, Proteobacteria, Clostridia, or a mixture thereof. In some embodiments, the one or more types of spore-forming bacteria comprise Clostridia Cluster IV bacteria, Clostridia Cluster XIVa bacteria, or both. In some embodiments, the composition comprising one or more types of spore-forming bacteria comprises spore-forming microbes from a human intestine. In some embodiments, the composition comprising one or more types of spore-forming bacteria comprises spore-forming microbes from a healthy human colon or small intestine. In some embodiments, at least 50% of the bacteria in the composition comprising one or more types of spore-forming bacteria are Clostridial species. In some embodiments, the composition comprising one or more types of spore-forming bacteria is a probiotic composition, a neutraceutical, a pharmaceutical composition, or a mixture thereof.

Further disclosed herein is a method for treating a disorder caused by serotonin deficiency. The method includes, in some embodiments, adjusting the level of one or more serotonin-related metabolites in a subject suffering from a disorder caused by serotonin deficiency, and thereby increasing the colonic or blood level of serotonin in the subject.

In some embodiments, the one or more serotonin-related metabolites comprise at least one of the metabolites listed in Table 1. In some embodiments, the one or more serotonin-related metabolites comprise at least one of deoxycholate, α-tocopherol, paminobenzoate, and tyramine. In some embodiments, the disorder is irritable bowel syndrome, inflammatory bowel disease, cardiovascular disease, osteoporosis, abnormal gastrointestinal motility, abnormal platelet aggregation, abnormal platelet activation, abnormal immune response, depression, anxiety, or a combination thereof.

### BRIEF DESCRIPTION OF THE DRAWINGS

FIG. 1 is a schematic illustration showing how the indigenous spore-forming microbes from the gut microbiota produce metabolites to promote host serotonin biosynthesis in the gastrointestinal tract and impact gastrointestinal motility and hemostasis.

FIGS. 2A-2D show that the gut microbiota modulates host peripheral serotonin levels. FIG. 2A depicts levels of 5-HT in sera from SPF, GF, conventionalized GF and antibiotic-treated SPF mice (P0, P21, P42=postnatal day of conventionalization or initiating antibiotic treatment; mice were sacrificed on postnatal day 56 (P56)). Data are normalized to serum 5-HT concentrations in SPF mice. n=8-13. FIG. 2B shows the levels of colon 5-HT relative to total protein content in colons from SPF, GF, conventionalized GF and antibiotic-treated SPF mice. Data are normalized to colon 5-HT levels relative to total protein content in SPF mice. n=8-13. FIG. 2C shows the expression of TPH1 relative to GAPDH in colons of SPF, GF, conventionalized GF and antibiotic-treated SPF mice. Data are normalized to expression levels in SPF mice. n=4. FIG. 2D shows the expression of SLC6A4 relative to GAPDH in colons of SPF, GF, conventionalized GF and antibiotic-treated SPF mice. Data are normalized to expression levels in SPF mice. n=4. The data presented in FIGS. 2A-2D are presented as mean±SEM. *p<0.05, **p<0.01, ***p<0.001, n.s.=not statistically significant. SPF=specific pathogen-free (conventionally-colonized), GF=germ-free, CONV.=SPF conventionalized, ABX=antibiotic-treated, VEH=vehicle (water)-treated.

FIGS. 3A-3I show the microbiota-dependent effects on serotonin metabolism. FIG. 3A depicts levels of 5-HT in adult SPF vs. GF mice. Data from colon and small intestine are normalized to total protein content. Colon: n=29-33, small intestine: n=6, feces: n=4, serum: n=12, platelet-rich plasma: n=6. FIG. 3B shows cecal weight after conventionalization of GF mice on postnatal day (P) 0, P21 and P42, and after antibiotic treatment of SPF mice on P0, P21 and P42. n=8-13. FIG. 3C shows the expression of genes involved in 5-HT metabolism relative to GAPDH in colons of adult SPF and GF mice. Data for each gene are normalized to expression levels in SPF mice. n=5. FIG. 3D shows the expression of TPH1 relative to GAPDH in distal, medial and proximal colons of adult SPF and GF mice. Data are normalized to expression levels in distal colon of SPF mice. n=5. FIG. 3E shows the expression of SLC6A4 relative to GAPDH in distal, medial and proximal colons of adult SPF and GF mice. Data are normalized to expression levels in distal colon of SPF mice. n=5. FIG. 3F shows the expression of neural-specific isoforms of genes involved in 5-HT metabolism relative to GAPDH in colons of adult SPF and GF mice. Data for each gene are normalized to expression levels in SPF mice. n=5. FIG. 3G shows mouse consumption of water supplemented with Trp (1.5 mg/ml) or 5-HTP (1.5 mg/ml). n=4. FIG. 3H shows the levels of colon 5-HT relative to total protein content two weeks after Trp or 5-HTP supplementation. Data are normalized to 5-HT levels in SPF mice. n=4-7. FIG. 3I shows the levels of serum 5-HT two weeks after Trp or 5-HTP supplementation. Data are normalized to 5-HT levels in SPF mice. n=4-7. The data shown in FIGS. 3A-3I are presented as mean±SEM. *p<0.05, **p<0.01, ***p<0.001, ****p<0.0001, n.s.=not statistically significant. SPF=specific pathogen-free (conventionally-colonized), GF=germ-free, CONV.=SPF conventionalized, ABX=antibiotic-treated, AADC=aromatic amino acid decarboxylase, Tph=tryptophan hydroxylase, Vmat=vesicular monoamine transporter, SERT=serotonin transporter (Slc6a4), MAO=monoamine oxidase, Lrp=lipoprotein receptor related protein, 5-HTP=5-hydroxytryptophan, Trp=tryptophan.

FIGS. 4A-4D show that indigenous spore-forming bacteria increase 5-HT levels in colon enterochromaffin cells (EC). FIG. 4A provides representative images of chromagranin A (CgA) (left), 5-HT (center), and merged (right) immunofluorescence staining in colons from SPF, GF, P42 GF-conventionalized, P42 antibiotictreated, and P42 spore-forming bacteria-colonized mice. Arrows indicate CgA-positive cells that lack 5-HT staining. n=3-7 mice/group. FIG. 4B shows quantitation of 5-HT+ cell number per area of colonic epithelial tissue. n=3-7 mice/group. FIG. 4C shows quantitation of CgA+ cell number per area of colonic epithelial tissue. n=3-7 mice/group. FIG. 4D shows the ratio of 5-HT+ cells/CgA+ cells per area of colonic epithelial tissue. n=3-7 mice/group. The data presented in FIGS. 4A-4D are presented as mean±SEM. *p<0.05, **p<0.01, ***p<0.001, ****p<0.0001. SPF=specific pathogen-free (conventionally-colonized), GF=germ-free, CONV.=SPF conventionalized, ABX=antibiotic-treated, Sp=spore-forming bacteria, PCPA=parachlorophenylalanine.

FIGS. 5A-5G shows the characterization of serotonin modulation by spore-forming bacteria. FIG. 5A shows the quantitation of 5-HT+ (left), CgA+ (center) and ratio of 5-HT+ cells/CgA+ cells per area of small intestinal epithelial tissue. n=3 mice/group. FIG. 5B provides representative images of CgA (left), 5-HT+ (center), and merged (right) immunofluorescence staining in small intestines from SPF and GF mice. n=3 mice/group. FIG. 5C shows the levels of serum 5-HT after intrarectal administration of PCPA or vehicle. n=4-7. FIG. 5D shows the expression of SLC6A4 relative to GAPDH in colons SPF, GF and Sp-colonized mice after treatment with PCPA or vehicle. Data are normalized to expression levels in SPF mice. n=3. FIG. 5E shows the levels of serum 5-HT at 2-14 days post treatment with mouse chloroform-resistant bacteria (spores, Sp). SPF: pooled from n=6, GF: pooled from n=6, GF+Sp: n=3-6. FIG. 5F shows cecal weight in SPF, GF, and P42 Sp-colonized mice. n=9-10. FIG. 5G shows the levels of colon 5-HT in SPF, GF and P42 Sp-colonized Rag1 KO mice. Data are normalized to levels in SPF mice. n=3. The data shown in FIGS. 5A-5G are presented as mean±SEM. *p<0.05, **p<0.01, ****p<0.0001. SPF=specific pathogen-free (conventionally-colonized), GF=germ-free, Sp=spore-forming bacteria, Rag=recombination activating gene, PCPA=para-chlorophenylalanine.

FIGS. 6A-6D shows that indigenous spore-forming bacteria induce colon 5-HT biosynthesis and systemic 5-HT bioavailability. FIG. 6A shows levels of serum 5-HT in gnotobiotic mice and controls. Data are normalized to serum 5-HT levels in SPF mice. SPF, n=13; GF, n=17; GF+conv.=P21 conventionalization, n=4; SPF+Abx=P42 antibiotic treatment, n=7; B. fragilis monoassociation (BF), n=6; SFB=Segmented Filamentous Bacteria monoassociation, n=4; ASF=Altered Schaedler Flora P21 colonization, n=4; Sp=spore-forming bacteria, P21 colonization, n=4; B. uniformis P21 colonization, n=4; Bd=Bacteroides consortium, n=3. FIG. 6B shows the levels of colon 5-HT relative to total protein content in gnotobiotic mice and controls. Data are normalized to colon 5-HT levels relative to total protein content in SPF mice. n=5-15. FIG. 6C shows the levels of colon 5-HT relative to total protein content in SPF, GF and Sp-colonized mice after intrarectal treatment with the Tph inhibitor, PCPA, or vehicle. n=4. FIG. 6D shows the expression of TPH1 relative to GAPDH in colons of SPF, GF and Sp-colonized mice after treatment with PCPA or vehicle. Data are normalized to expression levels in SPF mice. n=3. The data from FIGS. 6A-6D are presented as mean±SEM. *p<0.05, **p<0.01, ***p<0.001, ****p<0.0001, n.s.=not statistically significant. SPF=specific pathogen-free (conventionally-colonized), GF=germ-free, Sp=spore-forming bacteria, PCPA=para-chlorophenylalanine.

FIGS. 7A-7D shows that spore-forming bacteria from the healthy human gut microbiota promote colon 5-HT biosynthesis and systemic 5-HT bioavailability. FIG. 7A shows the levels of 5-HT relative to total protein content in colons from P56 SPF, GF, conventionalized GF and antibiotic-treated SPF mice. Data are normalized to colon 5-HT levels relative to total protein content in SPF mice. n=3-8. FIG. 7B shows the levels of 5-HT in sera from P56 SPF, GF, conventionalized GF and antibiotic-treated SPF mice. Data are normalized to scrum 5-HT concentrations in SPF mice. n=3-8. FIG. 7C shows the quantitation of 5-HT+ (left), CgA+ (center) and ratio of 5-HT+ to CgA+ cell number per area of colonic epithelial tissue. n=3-7 mice/group. FIG. 7D shows the representative images of chromagranin A (CgA) (left), 5-HT (center), and merged (right) immunofluorescence staining in colons from SPF, GF, P42 human spore-forming bacteria-colonized mice. Arrows indicate CgA-positive cells that lack 5-HT staining, n=3-7 mice/group. The data from FIGS. 7A-7D are presented as mean±SEM. *p<0.05, **p<0.01, ***p<0.001, ****p<0.0001, n.s.=not statistically significant. SPF=specific pathogen-free (conventionally-colonized), GF=germ-free, hSp=human-derived spore-forming bacteria, PCPA=para-chlorophenylalanine.

FIGS. 8A-8F shows that microbiota-mediated regulation of host serotonin modulates gastrointestinal motility. FIG. 8A shows the total time for transit of orally administered carmine red solution through the GI tract. n=4-8. FIG. 8B depicts the defecation rate as measured by number of fecal pellets produced relative to total transit time. n=4-8. FIG. 8C shows representative images of c-fos and 5HT4 colocalization in the colonic submucosa and muscularis externa. n=4-5 mice/group. FIG. 8D shows the quantitation of total c-fos fluorescence intensity in the colonic submucosa and muscularis externa. n=4-5 mice/group. FIG. 8E shows the quantitation of total 5HT4 fluorescence intensity in the colonic submucosa and muscularis externa. n=4-5 mice/group. FIG. 8F shows the quantitation and representative images of c-fos and calb2 (calretinin) colocalization in the colonic submucosa and muscularis externa. n=5-8 mice/group. The data from FIGS. 8A-8F are presented as mean±SEM. *p<0.05, **p<0.01, ***p<0.001, ****p<0.0001. SPF=specific pathogen-free (conventionally-colonized), GF=germ-free, Sp=spore-forming bacteria, PCPA=para-chlorophenylalanine.

FIGS. 9A-9E shows that microbiota modulates gastrointestinal 5-HT in the context of serotonin transporter gene deficiency. FIG. 9A shows the levels of 5-HT relative to total protein content in colons from SLC6A4 wild-type (+/+), heterozygous (+/−) and knockout (−/−) mice, treated with vehicle (water), antibiotics (Abx), or Abx+colonization with spore-forming bacteria (Sp). Data are normalized to colon 5-HT levels relative to total protein content in vehicle-treated (SPF) SLC6A4+/+ mice. n=5-8. FIG. 9B shows the levels of 5-HT in sera from SLC6A4+/+, +/− or −/−mice, treated with vehicle, Abx, or Abx and Sp. Data are normalized to serum 5-HT concentrations in vehicle-treated SLC6A4+/+ mice. n=5-8.

FIG. 9C shows representative images of chromagranin A (CgA) (left), 5-HT (center), and merged (right) immunofluorescence staining in colons from SLC6A4−/−mice, treated with vehicle, Abx, or Abx and Sp, relative to SLC6A4+/+SPF controls. Arrows indicate CgA-positive cells that lack 5-HT staining, n=3-7 mice/group. FIG. 9D shows the quantitation of 5-HT+ (left), CgA+ (center) and ratio of 5-HT+ to CgA+ cell number per area of colonic epithelial tissue from SLC6A4−/−mice, treated with vehicle, Abx, or Abx and Sp, relative to SLC6A4+/+SPF controls. n=3-7 mice/group. FIG. 9E shows the total time for transit of orally administered carmine red solution through the GI tract. n=5-8. The data of FIGS. 9A-9E are presented as mean±SEM. *p<0.05, **p<0.01, ***p<0.001, ****p<0.0001, n.s.=not statistically significant. SPF=specific pathogen-free (conventionally-colonized), Veh=vehicle (water), Abx=antibiotics, Sp=spore-forming bacteria

FIGS. 10A-10F shows that microbiota-mediated regulation of host serotonin modulates hemostasis. FIG. 10A shows the time to cessation of bleeding in response to tail injury in SPF, GF and Sp-colonized mice after treatment with PCPA or vehicle. n=7-16. FIG. 10B shows platelet activation, as measured by percentage of large, high granularity (FSChigh, SSChigh) events after collagen stimulation relative to unstimulated controls. Representative flow cytometry plots are shown in Figure D. n=3. FIG. 10C shows the representative flow cytometry plots of large, high granularity (FSChigh, SSChigh) activated platelets after collagen stimulation (bottom), as compared to unstimulated controls (top). n=3. FIG. 10D shows geometric mean fluorescence intensity of granulophysin (CD63) expression in collagen-stimulated platelets from SPF, GF and Sp-colonized mice after treatment with PCPA or vehicle (left). Representative flow cytometry histograms (right) showing event count vs. CD63 fluorescence intensity (log scale) for platelets treated with collagen (+) or vehicle (−). n=3. FIG. 10E shows geometric mean fluorescence intensity of P-selectin expression in collagen-stimulated platelets from SPF, GF and Sp-colonized mice after treatment with PCPA or vehicle (left). Representative flow cytometry histograms (right) showing event count vs. P-selectin fluorescence intensity (log scale) for platelets treated with collagen (+) or vehicle (−). n=3. FIG. 10F shows geometric mean fluorescence intensity of JON/A (integrin α∥bβ3) expression in collagen-stimulated platelets from SPF, GF and Sp-colonized mice after treatment with PCPA or vehicle (left). Representative flow cytometry histograms (right) showing event count vs. JON/A fluorescence intensity (log scale) for platelets treated with collagen (+) or vehicle (−). n=3. The data for FIGS. 10A-10F for platelet activation and aggregation assays are representative of three independent trials with at least three mice in each group. Data are presented as mean±SEM. *p<0.05, **p<0.01, ***p<0.001, ****p<0.0001, n.s.=not statistically significant. SPF=specific pathogen-free (conventionally-colonized), GF=germ-free, Sp=spore-forming bacteria, PCPA=parachlorophenylalanine.

FIGS. 11A-11D shows microbiota effects on platelet aggregation. FIG. 11A shows platelet counts from SPF, GF and Sp-colonized mice after treatment with PCPA or vehicle. n=3-7. FIG. 11B shows representative images of platelets after treatment with collagen (bottom) or vehicle (top). n=3. FIG. 11C shows platelet aggregation, as measured by percentage of large, high granularity CD9-APC^{mid}, CD9-Pe^{mid} Ter119− events, after collagen stimulation. Relative flow cytometry plots are shown in panel E. n=3. FIG. 11D shows representative flow cytometry plots of large, high granularity (FSChigh, SSChigh; events are circled) CD9-APCmid, CD9-PEmid aggregated platelets after collagen stimulation (bottom), as compared to unstimulated controls (top). n=3. The data of FIGS. 11A-11D for platelet activation and aggregation assays are representative of three independent trials with at least three mice in each group. Data are presented as mean±SEM. n.s.=not statistically significant. SPF=specific pathogen-free (conventionally-colonized), GF=germ-free, Sp=spore-forming bacteria, PCPA=para-chlorophenylalanine.

FIGS. 12A-12G shows that microbial metabolites mediate microbiota effects on host serotonin. FIG. 12A shows the levels of 5-HT released from RIN14B cells after exposure to colonic luminal filtrate from SPF, GF and Sp-colonized mice, or to the calcium ionophore, ionomycin (iono), as a positive control. Data are normalized to 5-HT levels in vehicle-treated RIN14B controls (hatched gray line at 1). Asterisks directly above bars indicate significance compared to vehicle-treated RIN14B controls, whereas asterisks at the top of the graph denote statistical significance between experimental groups. n=3. FIG. 12B shows the expression of TPH1 relative to GAPDH in RIN14B cells after exposure to colon luminal filtrate from SPF, GF and Sp-colonized mice, or ti the calcium ionophore, ionomycin (iono), as a positive control. Data are normalized to gene expression in vehicle-treated RIN14B controls (hatched gray line at 1). Asterisks directly above bars indicate significance compared to vehicle-treated RIN14B controls, whereas asterisks at the top of the graph denote statistical significance between experimental groups. n=4. FIG. 12C provides a principal components analysis of the fecal metabolome from GF mice colonized with SPF, ASF, Sp, or hSp n=6.

FIG. 12D shows the levels of 5-HT released from RIN14B cells after exposure to select metabolites identified to be commonly induced by SPF, Sp and hSp and to correlate positively with 5-HT levels: acetate (1 mM), α-tocopherol (8 uM), arabinose (50 uM), azelate (50 uM), butyrate (100 uM), cholate (75 uM), deoxycholate (25 uM), ferulate (25 uM), GABA (25 uM), glycine (50 uM), N-methyl proline (0.5 uM), oleanolate (50 uM), p-aminobenzoate (1 uM), propionate (100 uM), taurine (50 uM), tyramine (100 uM). Data are normalized to 5-HT levels in vehicle-treated RIN14B controls (gray line at 1). Asterisks directly above bars indicate significance compared to vehicle-treated RIN14B controls. n=5-19. FIG. 12E shows the expression of TPH1 relative to GAPDH in RIN14B cells after exposure to metabolites modulated by SPF, Sp and hSp, or to the calcium ionophore, ionomycin, as a negative control. Data are normalized to gene expression in vehicle-treated RIN14B controls (gray line at 1). Asterisks directly above bars indicate significance compared to vehicle-treated RIN14B controls. n=3-4. FIG. 12F shows the levels of 5-HT in colons (left) and serum (center) of GF C57B1/6 mice at 30 min after intrarectal injection of deoxycholate (125 mg/kg) or vehicle. Expression of TPH1 relative to GAPDH (right) at 1 hr post injection. n=3-8. FIG. 12G depicts a phylogenetic tree displaying key Sp. (M) and hSp. (H) operational units (OTUs) relative to reference Clostridium species with reported 7α-dehydroxylation activity (circles). Relative abundances of OTUs are indicated in parentheses. Select Bacteroides species that have no effect on colon and serum 5-HT levels (FIGS. 6A-6D) are included. Tree is based on nearest neighbor analysis of 16S rRNA gene sequences from fecal samples of mice colonized with Sp (n=3). The data of FIGS. 12A-12G are presented as mean±SEM. *p<0.05, **p<0.01, ***p<0.001, ****p<0.0001, n.s.=not statistically significant. SPF=specific pathogen-free (conventionally-colonized), GF-germ-free, Sp=spore-forming bacteria, iono=15 uM ionomycin, ASF=Altered Schaedler Flora, hSp=human-derived spore-forming bacteria.

FIGS. 13A-13E shows the metabolite effects on host 5-HT-related phenotypes. FIG. 13A shows the relative levels of 5-HT and additional metabolites that co-vary with 5-HT in colonic luminal contents from SPF, GF, Sp, ASF and hSp-colonized mice. a.u.=arbitrary units. n=6. FIG. 13B shows the levels of serum 5-HT (left) and colon 5-HT (right) in adult GF Swiss Webster mice at 1 hour after intrarectal injection with α-tocopherol (2.25 mg/kg), p-aminobenzoate (PABA; 1.37 μg/kg), tyramine (0.137 mg/kg), oleanolate (0.46 mg/kg) or vehicle. Data are normalized to 5-HT levels from GF mice injected with vehicle. n=5-8. FIG. 13C shows the levels of serum 5-HT (left) and colon 5-HT (right) in GF Swiss Webster mice intrarectally injected with α-tocopherol (2.25 mg/kg), deoxycholate (125 mg/kg), oleanolate (0.457 mg/kg) or vehicle. Data are normalized to serum 5-HT levels at 30 min after injection of GF mice with vehicle. n=2-5. FIG. 13D shows the total time for transit of orally administered carmine red solution through the GI tract in GF C57B1/6 mice intrarectally injected with α-tocopherol (2.25 mg/kg) or deoxycholate (125 mg/kg) and co-injection of PCPA or vehicle. n=3. FIG. 13E shows platelet activation, as measured by percentage of large, high granularity (FSChigh, SSChigh) events after collagen stimulation relative to unstimulated controls. n=3. The data of FIGS. 13A-13E are presented as mean±SEM. *p<0.05, **p<0.01, ***p<0.001, ****p<0.0001, n.s.=not statistically significant. SPF=specific pathogen-free (conventionally-colonized), GF=germ-free, PCPA=parachlorophenylalanine

FIG. 14 provides a phylogenetic analysis of OTUs from the feces of mice colonized with indigenous spore-forming bacteria phylogenetic tree, based on nearest-neighbor analysis of 16S rRNA gene sequences from fecal samples of mice colonized with Sp (M, n=3) or hSp (H, n=4), displaying Sp and hSp operational taxonomic units (OTUs) relative to reference species with reported 7α-dehydroxylation activity (highlighted, including Clostridium hiranonis, Clostridium leptum, Clostridium hylemonde, and Clostridium scindens) or gene homology to enzymes involved in metabolism of α-tocopherol (tocopherol o-methyltransferase), tyramine (tyrosine decarboxylase) and serotonin (among other monomines, monoamine oxidase). Relative abundances of OTUs are indicated in parentheses. Select Bacteroides species found to have no effect on colon and serum 5-HT levels (FIG. 6A) are included.

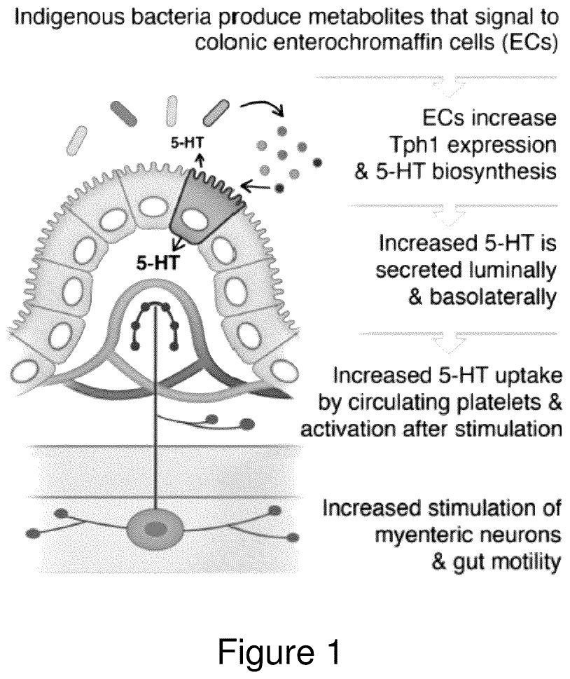

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Serotonin (5-hydroxytryptamine (5-HT)) is a tryptophan-derived monoamine neurotransmitter. It is primarily found in the gastrointestinal tract (GI tract), blood platelets, and the central nervous system (CNS) of animals, including humans. Serotonin has been found to regulate a variety of biological processes, for example intestinal movements, platelet activation/aggregation, stimulation of myenteric neurons and gut mobility, mood, appetite, sleep, some cognitive functions such as memory and learning, bone metabolism, and cardiac functions. Abnormal level of serotonin can cause various pathological conditions in animals.

As described herein, microbiota (e.g., gut microbiota) plays an important role in regulating the level of serotonin in the host. The composition of gut microbiota of a subject (e.g., a subject who suffers from or is at a risk of developing a serotonin-related disease) can be adjusted to modulate the level of serotonin in the subject. Also as described herein, various metabolites have the ability to modulate serotonin level in subjects. The level of serotonin-related metabolites in a subject with abnormal serotonin level may be adjusted to restore the level of serotonin to normal in the subject. In some embodiments, the level of one or more serotonin-related metabolites can be adjusted to modulate serotonin biosynthesis in a subject. Methods for treating serotonin-related diseases (e.g., a disorder caused by serotonin deficiency) are also disclosed herein. In some embodiments, the methods include adjusting the composition of gut microbiota in a subject suffering from one or more serotonin-related diseases, and increasing the colonic or blood level of serotonin in the subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, NY 1989). For purposes of the present disclosure, the following terms are defined below.

As used herein, the term “subject” is an animal, such as a vertebrate, preferably a mammal. The term “mammal” is defined as an individual belonging to the class Mammalia and includes, without limitation, humans, domestic and farm animals, and zoo, sports, or pet animals, such as sheep, dogs, horses, cats or cows. In some embodiments, the subject is mouse or rat. In some embodiments, the subject is human.

As used herein, the term “serotonin-related disease” refers to a condition, disease, disorder or symptom expressed by a subject having an abnormal serotonin level, for example a subject that has serotonin deficiency or has an excessive level of serotonin. The serotonin-related disease can be, or has a symptom of, an abnormality in enteric motor and/or secretory reflexes, an abnormality in platelet aggregation, an abnormality in immune responses, an abnormality in bone development, an abnormality in cardiac function, an abnormality in gastrointestinal motility, an abnormality in hemostasis, an abnormality in mood or cognition, an abnormality in osteoblast differentiation, an abnormality in hepatic regeneration, an abnormality in erythropoiesis, an abnormality in intestinal immunity, an abnormality in neurodevelopment, or any combination thereof. Examples of serotonin-related diseases include, but are not limited to, irritable bowel syndrome, inflammatory bowel disease, cardiovascular disease, osteoporosis, abnormal gastrointestinal motility, abnormal platelet aggregation, abnormal platelet activation, abnormal immune response, depression, anxiety, or a combination thereof. As used herein, the term “subject in need of the treatment” refers to a subject who is suffering from or at a risk of developing one or more of serotonin-related diseases.

As used herein, the term “treatment” refers to an intervention made in response to a disease, disorder or physiological condition manifested by a patient, particularly a patient suffering from one or more serotonin-related diseases. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may enhance or reduce the level of serotonin in the subject, thereby to reduce, alleviate, or eradicate the symptom(s) of the disease(s). As used herein, the term “prevention” refers to any activity that reduces the burden of the individual later expressing those serotonin-related disease symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipents which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The pharmaceutically acceptable or appropriate carrier may include other compounds known to be beneficial to an impaired situation of the GI tract, (e.g., antioxidants, such as Vitamin C, Vitamin E, Selenium or Zinc); or a food composition. The food composition can be, but is not limited to, milk, yoghurt, curd, cheese, fermented milks, milk based fermented products, ice-creams, fermented cereal based products, milk based powders, infant formulae, tablets, liquid bacterial suspensions, dried oral supplement, or wet oral supplement.

As used herein, the term “neutraceutical” refers to a food stuff (as a fortified food or a dietary supplement) that provides health benefits. Nutraceutical foods are not subject to the same testing and regulations as pharmaceutical drugs.

As used herein, the term “probiotic” refers to live microorganisms, which, when administered in adequate amounts, confer a health benefit on the host. The probiotics may be available in foods and dietary supplements (for example, but not limited to capsules, tablets, and powders). Non-limiting examples of foods containing probiotic include dairy products such as yogurt, fermented and unfermented milk, smoothies, butter, cream, hummus, kombucha, salad dressing, miso, tempeh, nutrition bars, and some juices and soy beverages.

As used herein, the term “metabolite” refers to any molecule involved in metabolism. Metabolites can be products, substrates, or intermediates in metabolic processes. For example, the metabolite can be a primary metabolite, a secondary metabolite, an organic metabolite, or an inorganic metabolite. Metabolites include, without limitation, amino acids, peptides, acylcarnitines, monosaccharides, oligosaccharides, lipids and phospholipids, prostaglandins, hydroxyeicosatetraenoic acids, hydroxyoctadecadienoic acids, steroids, bile acids, and glycolipids and phospholipids.

As used herein, the term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, and antibody fragments (e.g., Fab or F(ab′)₂, and Fv). For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6.

### Serotonin

The monoamine serotonin (5-hydroxytryptamine, 5-HT) is a well-known brain neurotransmitter. It is also an important regulatory factor in the GI tract and other organ systems. More than 90% of the body's 5-HT is synthesized in the gut, where 5-HT have been found to activate as many as 14 different 5-HT receptor subtypes located on various cell types including enterocytes, enteric neurons and immune cells). In addition, circulating platelets sequester 5-HT from the GI tract, releasing it to promote hemostasis and distributing it to various peripheral body sites. As such, gut-derived 5-HT plays a key role in regulating diverse biological processes, for example enteric motor and secretory reflexes, platelet aggregation, immune responses, erythropoiesis, bone development, cardiac function, and liver regeneration. Furthermore, dysregulation of peripheral 5-HT is implicated in the pathogenesis of various diseases, including but not limited to, irritable bowel syndrome (IBS), cardiovascular disease, and osteoporosis.

In the GI tract, 5-HT is synthesized independently by specialized endocrine cells, called enterochromaffin cells (ECs), as well as mucosal mast cells and myenteric neurons, which supply 5-HT to the mucosa, lumen and circulating platelets. In addition, two different isoenzymes of tryptophan hydroxylase (Tph), Tph1 and Tph2, mediate non-neuronal vs. neuronal 5-HT biosynthesis. Mechanostimulation of the intestinal epithelium and luminal application of pungent chemical stimuli, such as allyl isothiocyanate, cinnamaldehyde and caffeine, evoke 5-HT release from ECs. High concentrations of short chain fatty acids and glucose are also reported to stimulate 5-HT release from ECs.

Mammals are colonized by a vast and diverse collection of microbes that critically influences health and disease. As described herein, microbiota can be used to regulate blood 5-HT levels, wherein serum concentrations of 5-HT are dramatically reduced in mice reared in the absence of microbial colonization (germ-free, “GF”), compared to conventionally colonized (specific pathogen-free, “SPF”) controls. In addition, intestinal ECs are morphologically larger in GF vs. SPF rats, suggesting that host-microbe interactions can impact the development and/or function of 5-HT-producing cells.

As disclosed herein, the gut microbiota can regulate 5-HT biosynthesis from colonic ECs in a postnatally inducible and reversible manner. For example, spore-forming microbes (Sp) from the healthy mouse and human microbiota can be used to sufficiently mediate microbial effects on, for example, serum, colon and fecal 5-HT levels. Also shown herein, various fecal metabolites are elevated by indigenous spore-forming microbes and likely signal directly to colonic ECs to promote 5-HT biosynthesis. In addition, microbiota-mediated changes in colonic 5-HT regulate GI motility and blood hemostasis in the host, so that targeting the microbiota can be used for modulating peripheral 5-HT bioavailability and treating 5-HT-related disease symptoms.

### Methods for Modulating Serotonin Level in a Subject

Methods for modulating the serotonin level in a subject are provided herein. In some embodiments, the methods include adjusting the composition of gut microbiota in a subject, and thereby changing the level of serotonin in the subject. In some embodiments, the level of serotonin in the subject is increased. In some embodiments, the level of serotonin in the subject is reduced.

The methods, in some embodiments, include determining the level of serotonin in the subject before the composition of gut microbiota in the subject is adjusted, after the composition of gut microbiota in the subject is adjusted, or both. In some embodiments, the methods include identifying a subject that suffers from a serotonin-related disease or is at the risk of developing a serotonin-related disease.

The subject can be a subject suffers from or is at a risk of developing one or more serotonin-related diseases. Non-limited examples of serotonin-related disease include irritable bowel syndrome, inflammatory bowel disease, cardiovascular disease, osteoporosis, abnormal gastrointestinal (GI) motility, abnormal platelet aggregation, abnormal platelet activation, abnormal immune response, depression, anxiety, and any combination thereof. In some embodiments, the subject suffers from irritable bowel syndrome, cardiovascular disease, osteoporosis, or a combination thereof. In some embodiments, the subject suffers from abnormal GI motility. In some embodiments, the subject suffers from an abnormality in enteric motor and secretory reflexes, an abnormality in platelet aggregation, an abnormality in immune responses, an abnormality in bone development, an abnormality in cardiac function, an abnormality in gastrointestinal motility, an abnormality in hemostasis, an abnormality in mood, an abnormality in cognition, an abnormality in osteoblast differentiation, an abnormality in hepatic regeneration, an abnormality in erythropoiesis, an abnormality in intestinal immunity, an abnormality in neurodevelopment, or any combination thereof.

In some embodiments, adjusting the composition of gut microbiota increases the expression of TPH1 gene in the subject. In some embodiments, adjusting the composition of gut microbiota promotes serotonin biosynthesis from intestinal enterochromaffin cells (e.g., colonic enterochromaffin cells) in the subject. In some embodiments, adjusting the composition of gut microbiota reduces or inhibits serotonin biosynthesis from intestinal enterochromaffin cells (e.g., colonic enterochromaffin cells) in the subject.

The methods disclosed herein can be used to change various level of serotonin in the subject. For example, changing the level of serotonin in the subject can include changing one or more of the gut level, the colonic level, the peripheral level, the serum level, the plasma level, and the fecal level of serotonin in the subject. In some embodiments, the methods change the gut level of serotonin. In some embodiments, the methods change the blood or colonic level of serotonin. As disclosed herein, adjusting the composition of gut microbiota can impact various biological processes in the subject, for example enhancing one or more of gastrointestinal motility, platelet activation, and platelet aggregation of the subject.

Various methods can be used to adjust the composition of gut microbiota of the subject. For example, adjustment of the composition of gut microbiota in the subject can be achieved by, for example, fecal transplantation (also known as fecal microbiota transplantation (FMT), fecal bacteriotherapy or stool transplant). Fecal transplantation can include a process of transplantation of fecal bacteria from a healthy donor, for example a subject that does not have, or not at a risk of developing, a serotonin-related disease, to a recipient (e.g., a subject suffering from, or at a risk of developing, a serotonin-related disease). The procedure of fecal transplantation can include single or multiple infusions (e.g., by enema) of bacterial fecal flora from the donor to the recipient. In addition, adjustment of the composition of gut microbiota in the subject can be achieved by microbiota conventionalization, microbial colonization, reconstitution of gut microbiota, probiotic treatment, antibiotic treatment, or a combination thereof. The method may, or may not, include additional therapeutically treatment. For example, in some embodiments, the methods do not include antibiotic treatment.

### Composition Comprising Spore-Forming Bacteria and Administration Thereof

Disclosed herein are compositions containing one or more types of spore-forming bacteria. In some embodiments, the composition does not comprise any pharmaceutically active ingredients, for example antibiotics, antidepressants, pain medications, selective serotonin reuptake inhibitors (SSRI drugs), and monoamine oxidase inhibitors (MAO inhibitors). For example, the composition may not contain any antibiotics. In some embodiments, the composition only contains one type of spore-forming bacteria, for example Clostridia bacteria. The composition can, in some embodiments, contain only bacteria from two, three, or four genus. The composition may, or may not, contain any prebiotics.

The one or more types of spore-forming bacteria can, for example, include Lactobacillales, Proteobacteria, Clostridia, or a mixture thereof. In some embodiments, at least, or at least about, 20%, 30%, 40%, 50%, 60%, 70%, 80%, 90%, 95%, 98%, or more, of the bacteria in the composition comprising one or more types of spore-forming bacteria are Lactobacillales, Proteobacteria, Clostridia, or a mixture thereof. In some embodiments, 20%, 30%, 40%, 50%, 60%, 70%, 80%, 90%, 95%, 98%, or a value between any two of these values (including end points), of the bacteria in the composition comprising one or more types of spore-forming bacteria are Lactobacillales, Proteobacteria, Clostridia, or a mixture thereof. The amount of the total bacteria and/or any specific bacteria contained in the composition can be determined by any conventional methods known in the art. For example, the amount of bacteria can be determined by measuring colony-forming unit (CFU or cfu) in a given amount of the composition. In general, if the composition is a liquid, the result is given as CFU/ml; and if the composition is a solid, the result is given as CFU/g. In another method, the bacteria contained in the composition can be isolated using the method described in Vaahtovuo et al. J Microbiol Methods. 63:276-286 (2005), and the bacteria can be fixed to determine the bacteria amount.

As another example, the one or more types of spore-forming bacteria can comprise Clostridia Cluster IV species, Clostridia Cluster XIVa species, or both. In some embodiments, the composition comprising one or more types of spore-forming bacteria comprises spore-forming microbes from a human intestine, for example a healthy human colon or small intestine. The composition comprising one or more types of spore-forming bacteria can be dominated by Clostridial species. Non-limiting examples of Clostridial species include Clostridium hiranonis, Clostridium leptum, Clostridium hylemonde, and Clostridium scindens. For example, in some embodiments, at least, or at least about, 20%, 30%, 40%, 50%, 60%, 70%, 80%, 90%, 95%, 98%, or more, of the bacteria in the composition are Clostridial species. In other words, the proportion of Clostridia species in relation to the total bacteria in the composition is at least, or at least about, 20%, 30%, 40%, 50%, 60%, 70%, 80%, 90%, 95%, 98% or more. In some embodiments, about 20%, 30%, 40%, 50%, 60%, 70%, 80%, 90%, 95%, 98%, or a value between any two of these values (including end points), of the bacteria in the composition comprising one or more types of spore-forming bacteria are Clostridial species. In some embodiments, 20%, 30%, 40%, 50%, 60%, 70%, 80%, 90%, 95%, 98%, or a value between any two of these values (including end points), of the bacteria in the composition comprising one or more types of spore-forming bacteria are Clostridial species. In some embodiments, at least 50% of the bacteria in the composition are Clostridial species. In some embodiments, at least, or at least about, 20%, 30%, 40%, 50%, 60%, 70%, 80%, 90%, 95%, 98%, or more, of the bacteria in the composition comprising one or more types of spore-forming bacteria are Clostridia Cluster IV, Clostridia Cluster XIVa species, or a mixture thereof. In some embodiments, 20%, 30%, 40%, 50%, 60%, 70%, 80%, 90%, 95%, 98% or a value between any two of these values (including end points) of the bacteria in the composition comprising one or more types of spore-forming bacteria are Clostridia Cluster IV, Clostridia Cluster XIVa species, or a mixture thereof.

The composition comprising one or more types of spore-forming bacteria may or may not contain bifidobacteria (also known as Lactobacillus bifidus). Non-limiting examples of bifidobacteria include Bifidobacterium longum, Bifidobacterium bifidum, Bifidobacterium breve, Bifidobacterium. infantis, Bifidobacterium animalis subsp. lactis Bb-12 and Bifidobacterium lactis B1. In some embodiments, the composition contains a small amount of bifidobacteria. For example, the composition can contain less than, or less than about, 0.1%, 0.5%, 1%, 2%, 5%, 10%, 15%, 20%, 25%, or 30%, of bifidobacteria. In some embodiments, the composition contains more Clostrial species than bifidobacteria. For example, the ratio between the amount of Clostrial species and the amount of bifidobacteria in the composition can be greater than, or greater than about, 1.5, 2, 2.5, 3, 4, 5, 8, 10, 15, 20, 25, 50, 100, 500, or 1000. In some embodiments, the composition does not comprise bifidobacteria. The composition comprising one or more types of spore-forming bacteria may or may not contain any lactic acid bacteria. Non-limiting examples of lactic acid bacterial include lactobacilli, for example Lactobacillus rhamnosus and Lactobacillus casei. In some embodiments, the composition contains a small amount of lactic acid bacteria, for example less than, or less than about, 0.1%, 0.5%, 1%, 2%, 5%, 10%, 15%, 20%, 25%, or 30% of lactic acid bacteria. In some embodiments, the composition does not contain lactic acid bacteria. In some embodiments, the composition contains more Clostrial species than lactic acid bacteria. For example, the ratio between the amount of Clostrial species and the amount of lactic acid bacteria in the composition can be greater than, or greater than about, 1.5, 2, 2.5, 3, 4, 5, 6, 7, 8, 10, 15, 20, 25, 50, 100, 500, or 1000.

In addition, the composition comprising one or more types of spore-forming bacteria may or may not contain Bacteroides species, including but not limited to, Bacteroides fragilis, Bacteroides uniformis, Bacteroides thetaiotaomicron, Bacteroides acidifaciens and Bacteroides vulgatus. In some embodiments, the composition contains a small amount of Bacteroides species. For example, less than, or less than about, 0.1%, 0.5%, 1%, 2%, 5%, 10%, 15%, 20%, 25%, or 30%, of the bacteria in the composition are Bacteroides species. In some embodiments, the composition contains much more Clostrial species than Bacteroides species. For example, the ratio between the amount of Clostrial species and the amount of Bacteroides species in the composition is greater than, or greater than about, 1.5, 2, 2.5, 3, 4, 5, 6, 7, 8, 10, 15, 20, 25, 50, 75, 100, 500, or 1000. In some embodiments, the composition does not contain Bacteroides species. In some embodiments, the composition does not comprise one or more of Bacteroides fragilis, Bacteroides uniformis, Bacteroides thetaiotaomicron, Bacteroides acidifaciens and Bacteroides vulgatus. In some embodiments, the composition does not comprise two or more, three or more, four or more of Bacteroides fragilis, Bacteroides uniformis, Bacteroides thetaiotaomicron, Bacteroides acidifaciens and Bacteroides vulgatus. In some embodiments, the composition does not comprise any of Bacteroides fragilis, Bacteroides uniformis, Bacteroides thetaiotaomicron, Bacteroides acidifaciens and Bacteroides vulgatus.

The composition comprising spore-forming bacteria can be in various forms, including but not limited to, a probiotic composition, a neutraceutical, a pharmaceutical composition, or a mixture thereof. In some embodiments, the composition is a probiotic composition. Each dosage for human and animal subjects preferably contains a predetermined quantity of the bacteria calculated in an amount sufficient to produce the desired effect. The actual dosage forms will depend on the particular bacteria employed and the effect to be achieved. The composition comprising spore-forming bacteria, for example, a composition comprising Clostridial bacteria, can be administered alone or in combination with one or more additional probiotic, neutraceutical, or therapeutic agents. Administration “in combination with” one or more additional probiotic, neutraceutical, or therapeutic agents includes both simultaneous (at the same time) and consecutive administration in any order. Administration can be chronic or intermittent, as deemed appropriate by the supervising practitioner, particularly in view of any change in the disease state or any undesirable side effects. “Chronic” administration refers to administration of the composition in a continuous manner while “intermittent” administration refers to treatment that is done with interruption. The composition comprising spore-forming bacteria can be administered with food or drink, for example, or separately in the form of a capsule, granulate, powder or liquid, for example.

The composition comprising spore-forming bacteria can also be administered to the subject via various routes, including but not limited to, oral administration, rectal administration, aerosol, parenteral administration, topical administration, subcutaneous administration, pulmonary administration, nasal administration, buccal administration, ocular administration, dermal administration, vaginal administration, intramuscular administration, or a combination thereof. For example, the composition can be administered to the subject via oral administration, rectum administration, transdermal administration, intranasal administration or inhalation. In some embodiments, the composition is administered to the subject orally. In some embodiments, the composition is administered to the colon of the subject.

In the methods described herein, adjusting the composition of gut microbiota of the subject can comprise administering to the subject a composition comprising one or more types of spore-forming bacteria. The composition comprising one or more types of spore-forming bacteria can be any of the spore-forming bacteria-containing composition disclosed herein. In some embodiments, the one or more types of spore-forming bacteria comprise Lactobacillales, Proteobacteria, Clostridia, or a mixture thereof. In some embodiments, the one or more types of spore-forming bacteria comprise Clostridia Cluster IV species, Clostridia Cluster XIVa species, or both. In some embodiments, the composition comprising spore-forming bacteria comprises spore-forming bacteria from a human intestine (e.g., colon or small intestine).

In some embodiments, adjusting the composition of gut microbiota in the subject includes reducing the level of one or more bacterial species in the subject. For example, the level of lactic acid bacteria in the subject can be reduced. In some embodiments, the lactic acid bacteria is lactobacilli (including, but not limited to, Lactobacillus rhamnosus and Lactobacillus casei). The level of bifidobacteria (including, but not limited to, Bifidobacterium longum, Bifidobacterium bifidum, Bifidobacterium breve, Bifidobacterium. infantis, Bifidobacterium animalis subsp. lactis Bb-12, and/or Bifidobacterium lactis B1) can also be reduced to adjust the composition of gut microbiota in the subject. Various methods can be used to reduce the level of one or more bacteria species in the subject. For example, a reduced carbohydrate diet, an antibiotic treatment, or both can be provided to the subject to reduce one or more intestinal bacterial species. Without being bound to any specific theory, it is believed that a reduced carbohydrate diet can restrict the available material necessary for bacterial fermentation to reduce intestinal bacterial species.

In some embodiments, adjust the composition of gut microbiota of the subject comprises administering the subject a composition comprising products derived from one or more types of spore-forming bacteria. Examples of products derived from bacteria include, but are not limited to, small molecules, polypeptides, lipids, enzymes, sugars, nucleic acids that are derived or produced from the bacteria, or any combination thereof.

In the methods disclosed herein, after adjustment of the composition of gut microbiota in the subject, the serotonin level in the subject can be, or be about, 40%, 50%, 60%, 70%, 80%, 90%, 95%, 98%, 99%, 100%, 101%, 102%, 105%, 110%, 120%, 130%, 140%, 150%, 200%, 250%, 300%, 400%, 500%, or a range between any two of these values, of the serotonin level in the subject prior to the adjustment. In some embodiments, the serotonin level in the subject after the adjustment of the composition of gut microbiota is at least, or is at least about, 50%, 60%, 70%, 80%, 85%, 90%, 95%, 98%, 99%, 100%, 101%, 102%, 105%, 110%, 120%, 130%, 150%, 200%, 300%, or 500%, of the serotonin level in the subject prior to the adjustment. In some embodiments, the serotonin level in the subject is no more than, or is no more than about, 50%, 60%, 70%, 80%, 90%, 95%, 98%, 99%, 100%, 101%, 102%, 105%, 110%, 150%, 200%, 300%, 400%, or 500%, of the serotonin level in the subject prior to the treatment.

The serotonin level in the subject can be restored to be, or be close to, normal serotonin level (e.g., the serotonin level in subjects that do not have, or are not at a risk of developing, serotonin-related diseases). One of skill in the art will appreciate that variability in serotonin level may exist between individuals, and a reference level can be established as a value representative of the serotonin level in a population of subjects that do not suffer from or at a risk of developing any serotonin-related disease or any pathological condition with one or more of the symptoms of the serotonin-related diseases, for the comparison. Various criteria can be used to determine the inclusion and/or exclusion of a particular subject in the reference population, including but not limited to, age of the subject (e.g. the reference subject can be within the same age group as the subject in need of treatment) and gender of the subject (e.g. the reference subject can be the same gender as the subject in need of treatment). In some embodiments, serotonin is at an increased level in the subjects suffering from one or more serotonin-related diseases as compared to the reference level. In some embodiments, serotonin is at a decreased level in the subjects suffering from one or more serotonin-related diseases as compared to the reference level. In some embodiments, the alteration in the level of serotonin can be restored partially or fully by adjusting the composition of gut microbiota in the subject suffering from one or more serotonin-related diseases.

For example, after adjustment of the composition of gut microbiota in the subject, the serotonin level in the subject can be, or can be about, 50%, 60%, 70%, 80%, 90%, 95%, 98%, 99%, 100%, 101%, 102%, 105%, 110%, 120%, 130%, 140%, 150%, 200%, or a range between any two of these values, of the reference serotonin level in subjects that do not have or are not at a risk of developing serotonin-related diseases. In some embodiments, the serotonin level in the subject is at least, or is at least about, 80%, 85%, 90%, 95%, 98%, 99%, 100%, 101%, 102%, 105%, 110%, 120%, 130%, 150%, of the reference serotonin level in subjects that do not have or are not at a risk of developing serotonin-related diseases. In some embodiments, the serotonin level in the subject is no more than, or is no more than about, 90%, 95%, 98%, 99%, 100%, 101%, 102%, 105%, or 110%, of the serotonin level in subjects that do not have, or are not at a risk of developing, serotonin-related diseases.

The serotonin level can be the level of serotonin in circulation of the subject. For example, the level of serotonin can be the peripheral level of serotonin, and/or the level of serotonin in blood or other body fluids (e.g., cerebrospinal fluid, pleural fluid, amniotic fluid, semen, or saliva) of the subject. In some embodiments, the level of serotonin is the fecal level of serotonin in the subject. In some embodiments, the level of serotonin is the blood level of serotonin in the subject. The blood level of serotonin can be, for example, serum level or plasma level of serotonin.

### Serotonin-Related Metabolites and the Use Thereof

As used herein, the term “serotonin-related metabolite” refers to a metabolite that has the ability to modulate serotonin level in vitro, ex vivo, and/or in vivo. In some embodiments, a serotonin-related metabolite co-varies with serotonin. For example, the metabolite may positively or negatively correlate with serotonin level. In some embodiments, the serotonin-related metabolite can promote serotonin biosynthesis, and thus increase the level of serotonin in the subject. In some embodiments, the serotonin-related metabolite can reduce or inhibit serotonin biosynthesis, and thus reduce the level of serotonin in the subject. The serotonin level can be, for example, the colonic serotonin level, the blood serotonin level, the peripheral serotonin level, the fecal serotonin level, or a combination thereof. In some embodiments, the serotonin-related metabolite can stimulate serotonin synthesis in and/or serotonin release from intestinal ECs.

For some serotonin-related metabolites, the level of the metabolite is altered in a subject having an abnormal level of serotonin as compared to subjects having a normal level of serotonin. For example, the level of the metabolite may be altered in circulation of the subject having an abnormal level of serotonin (e.g., a subject suffering from or at a risk of developing a serotonin-related disease) as compared to subjects having a normal level of serotonin. In some embodiments, the level of the metabolite is altered in blood, serum, plasma, body fluids (e.g., cerebrospinal fluid, pleural fluid, amniotic fluid, semen, or saliva), urine, and/or feces of the subject having an abnormal level of serotonin (e.g., a subject suffering from or at a risk of developing a serotonin-related disease) as compared to subjects having a normal level of serotonin. In some instances, the serotonin-related metabolite plays a causative role in the development of the serotonin-related disease in the subject. The serotonin-related metabolite can have an increased or decreased level in the subject having an abnormal level of serotonin as compared to subjects that does not suffer from any serotonin-related disease, or any pathological condition with one or more of the symptoms of serotonin-related diseases.

One of skill in the art will appreciate that variability in the level of metabolites may exist between individuals, and a reference level can be established as a value representative of the level of the metabolites in a population of subjects that do not suffer from any serotonin-related disease or any pathological condition with one or more of the symptoms of the serotonin-related diseases, for the comparison. Various criteria can be used to determine the inclusion and/or exclusion of a particular subject in the reference population, including but not limited to, age of the subject (e.g. the reference subject can be within the same age group as the subject in need of treatment) and gender of the subject (e.g. the reference subject can be the same gender as the subject in need of treatment). In some embodiments, the serotonin-related metabolite has an increased level in the subject suffering from one or more serotonin-related diseases as compared to the reference level. In some embodiments, the serotonin-related metabolite has a decreased level in the subject suffering from one or more serotonin-related diseases as compared to the reference level. In some embodiments, the alteration in the level of serotonin-related metabolite can be restored partially or fully by adjusting the composition of gut microbiota in the subject suffering from one or more serotonin-related diseases.

The level of the serotonin-related metabolite can be the level of the metabolite in circulation of the subject. For example, the level of the metabolite is the level of the metabolite in blood or other body fluids (e.g., cerebrospinal fluid, pleural fluid, amniotic fluid, semen, or saliva) of the subject. In some embodiments, the level of the metabolite is the blood level of the metabolite in the subject. The blood level of the metabolite can be, for example, serum level or plasma level of the metabolite. In some embodiments, the level of the metabolite is the fecal level of the metabolite in the subject. In some embodiments, the level of the metabolite is the colonic or peripheral level of the metabolite in the subject.

Non-limiting examples of serotonin-related metabolites are provided in Table 1.

TABLE 1
Exemplary serotonin-related metabolites

| | | | |
|---|---|---|---|
| ferulate | α-tocopherol | oleanolate | equol sulfate |
| N-methyl proline | sarcosine (N- | L-urobilin | pyridoxate (vitamin B6) |
| | Methylglycine) | | |
| ribulose | deoxycholate | cholate | alpha-muricholate |
| isoleucyltyrosine | cis-vaccenate (18:1n7) | hyocholate | 4-hydroxyphenylpyruvate |
| glycerol | 13-methylmyristic acid | N-acetyltaurine | azelate (nonanedioate) |
| arabinose | oleate (18:1n9) | D-urobilin | N-acetylneuraminate |
| rhamnose | linolenate [alpha or | guanine | coproporphyrin III |
| | gamma; (18:3n3 or 6)] | | |
| gluconate | N-acetylglucosamine | leucylglycine | sebacate (decanedioate) |
| taurine | asparagylleucine | equol | 4-methyl-2-oxopentanoate |
| pentadecanoate | 3-methyl-2-oxobutyrate | xylose | glutamine-leucine |
| (15:0) | | | |
| fructose | phenylalanylglycine | delta-tocopherol | 2-(4- |
| | | | hydroxyphenyl)propionate |
| adenine | serylleucine | valerate | glucosamine |
| allantoin | 3-methyl-2-oxovalerate | cystine | 1- |
| | | | linoleoylglycerophosphoethanolamine |
| xylitol | 3-phenylpropionate | vanillate | 2-methylbutyrylcarnitine |
| | (hydrocinnamate) | | (C5) |
| urate | 1- | pinitol | tauro(alpha + |
| | palmitoylglycerophosphoinositol | | beta)muricholate |
| myo-inositol | gamma- | creatine | tauroursodeoxycholate |
| | glutamylthreonine | | |
| threonate | pro-hydroxy-pro | asparagine | dimethylarginine (SDMA + |
| | | | ADMA) |
| arabonate | 2-palmitoylglycerol (2- | diaminopimelate | p-aminobenzoate |
| | monopalmitin) | | |
| taurocholate | 3-[3- | mannitol | soyasaponin II |
| urea | (sulfooxy)phenyl]propanoic | | |
| | acid | | |
| arabitol | gamma-glutamylvaline | palmitate (16:0) | glycylglycine |
| xylonate | soyasaponin I | tyramine | cysteine |

The serotonin-related metabolites are involved in various metabolic pathways. Examples of metabolic pathways that the serotonin-related metabolite can be involved in include, but are not limited to, amino acid metabolism, xenobiotics metabolism, peptide metabolism, carbohydrate metabolism, lipid metabolism, nucleotide metabolism, and metabolism of cofactors and vitamins. For example, the serotonin-related metabolite can be a metabolite involved in tryptophan metabolism; food component/plant metabolism; tocopherol metabolism; fatty acid metabolism (e.g., long chain fatty acid metabolism, short chain fatty acid metabolism, branched fatty acid metabolism, and polyunsaturated fatty acid metabolism); primary or secondary bile acid metabolism; arginine and proline metabolism; pentose metabolism; hemoglobin and porphyrin metabolism; dipeptide metabolism; glycerolipid metabolism; vitamin B6 metabolism; phenylalanine and tyrosine metabolism; methionine, cysteine, SAM and taurine metabolism; glycine, serine and threonine metabolism; aminosugar metabolism; fructose, mannose and galactose metabolism; leucine, isoleucine and valine metabolism; purine metabolism; purine metabolism; lysolipid metabolism; inositol metabolism; gamma-glutamyl amino acid metabolism; creatine metabolism; monoacylglycerol metabolism; or a combination thereof.

In some embodiments, the serotonin-related metabolite is a dipeptide. In some embodiments, the serotonin-related metabolite is a metabolite involved in glycolysis, gluconeogenesis, pyruvate metabolism, nucleotide metabolism, sugar metabolism, pentose metabolism, or a combination thereof. In some embodiments, the serotonin-related metabolite is a metabolite involved in essential fatty acid, long chain fatty acid, inositol, and/or lysolipid metabolism. In some embodiments, the serotonin-related metabolite is a metabolite involved in tocopherol metabolism. In some embodiments, the serotonin-related metabolite is a metabolite involved in secondary bile acid metabolism.

In some embodiments, the serotonin-related metabolite is a short chain fatty acid, acetate, butyrate, or propionate. In some embodiments, the serotonin-related metabolite is deoxycholate, α-tocopherol, tyramine, p-aminobenzoate, or any combination thereof. In some embodiments, the serotonin-related metabolite is deoxycholate. In some embodiments, the serotonin-related metabolite is α-tocopherol. In some embodiments, the serotonin-related metabolite is tyramine. In some embodiments, the serotonin-related metabolite is p-aminobenzoate.

Described herein are methods for modulating serotonin biosynthesis in a subject, comprising adjusting the level of one of the one or more serotonin-related metabolites in the subject. The one or more serotonin-related metabolites can, for example, comprise at least one of the metabolites listed in Table 1. In some embodiments, the method also includes determining the level of one or more serotonin-related metabolites in the subject.

In some embodiments, the level of at least one serotonin-related metabolite is adjusted to modulate serotonin biosynthesis in the subject. The metabolite level can be the blood level (e.g., serum and/or plasma level) and/or intestinal level (e.g., colonic level) of the metabolite. For example, the level of one or more of deoxycholate, α-tocopherol, tyramine and p-aminobenzoate in the subject can be adjusted to increase or decrease the serotonin level (e.g., blood and/or colonic level) in the subject. In some embodiments, the level of two or more serotonin-related metabolites is adjusted to modulate serotonin biosynthesis in the subject. For example, the level of two or more of deoxycholate, α-tocopherol, tyramine and p-aminobenzoate in the subject can be adjusted to increase or decrease the serotonin level (e.g., blood and/or colonic level) in the subject. In some embodiments, the level of deoxycholate is adjusted to modulate serotonin biosynthesis in the subject. In some embodiments, the level of α-tocopherol is adjusted to modulate serotonin biosynthesis in the subject. In some embodiments, the level of tyramine is adjusted to modulate serotonin biosynthesis in the subject. In some embodiments, the level of p-aminobenzoate is adjusted to modulate serotonin biosynthesis in the subject.

Various methods can be used to adjust the level, for example blood level (e.g., serum and/or plasma level) or intestinal level (e.g., colonic level), of the serotonin-related metabolite in the subject to modulate (e.g., increase or reduce) the serotonin level in the subject. For example, the level of the metabolite in the subject can be increased by administering the serotonin-related metabolite to the subject. The metabolite can be administered to the subject via a variety of route, including but not limited to, oral administration, rectal administration, aerosol, parenteral administration, topical administration, subcutaneous administration, pulmonary administration, nasal administration, buccal administration, ocular administration, dermal administration, vaginal administration, intramuscular administration, and a combination thereof. In another example, the level of the metabolite in the subject can be increased by activating an enzyme involved in the in vivo synthesis of the serotonin-related metabolite, administering such an enzyme to the subject, or both. As another example, the level of the metabolite in the subject can be increased by administering an intermediate or a substrate for the in vivo synthesis of the serotonin-related metabolite to the subject. The enzyme, the intermediate, or the substrate can also be administered to the subject, for example, via a variety of route, including but not limited to, oral administration, rectal administration, aerosol, parenteral administration, topical administration, subcutaneous administration, pulmonary administration, nasal administration, buccal administration, ocular administration, dermal administration, vaginal administration, intramuscular administration, and a combination thereof.

In some embodiments, the level of the serotonin-related metabolite in the subject can be reduced to modulate the serotonin level in the subject. For example, an antibody that specifically binds the metabolite, an antibody that specifically binds an intermediate for the in vivo synthesis of the metabolite, an antibody that specifically binds a substrate for the in vivo synthesis of the metabolite, or a combination thereof can be administered to the subject to adjust (e.g., increase or reduce) the level of the metabolite in the subject. For example, an antibody that specifically binds deoxycholate, an antibody that specifically binds one or more of the substrates and/or intermediates in the in vivo deoxycholate synthesis, or a combination thereof can be used to reduce the level of deoxycholate in the subject. As another example, an antibody that specifically binds α-tocopherol, an antibody that specifically binds one or more of the substrates and/or intermediates in the in vivo α-tocopherol synthesis, or a combination thereof can be used to reduce the level of α-tocopherol in the subject. As yet another example, an antibody that specifically binds tyramine, an antibody that specifically binds one or more of the substrates and/or intermediates in the in vivo tyramine synthesis, or a combination thereof can be used to reduce the level of tyramine in the subject. As still another example, an antibody that specifically binds p-aminobenzoate, an antibody that specifically binds one or more of the substrates and/or intermediates in the in vivo p-aminobenzoate synthesis, or a combination thereof can be used to reduce the level of p-aminobenzoate in the subject.

Methods for generating antibodies that specifically bind small molecules have been developed in the art. For example, generation of monoclonal antibodies against small molecules has been described in Rufo et al., J. Ag. Food Chem. 52:182-187 (2004), which is hereby incorporated by reference. For example, an animal such as a guinea pig or rat, preferably a mouse, can be immunized with a small molecule conjugated to a hapten (e.g., KLH), the antibody-producing cells, preferably splenic lymphocytes, can be collected and fused to a stable, immortalized cell line, preferably a myeloma cell line, to produce hybridoma cells which are then isolated and cloned. See, e.g., U.S. Pat. No. 6,156,882, which is hereby incorporated by reference. In addition, the genes encoding the heavy and light chains of a small molecule-specific antibody can be cloned from a cell, e.g., the genes encoding a monoclonal antibody can be cloned from a hybridoma and used to produce a recombinant monoclonal antibody.

The level, for example blood level (e.g., serum and/or plasma level) or intestinal level (e.g., colonic level), of the serotonin-related metabolite in the subject can also be reduced by inhibiting an enzyme involved in the in vivo synthesis of the metabolite to modulate serotonin level in the subject.

The level of the one or more serotonin-related metabolite(s) can also be adjusted by adjusting the composition of gut microbiota in the subject. As described above, various methods can be used to adjust the composition of gut microbiota of the subject, for example by administering the subject a composition comprising one or more types of spore-forming bacteria. As described above, the composition can comprise various types of spore-forming bacteria. For example, the one or more types of spore-forming bacteria can comprise Lactobacillales, Proteobacteria, Clostridia, or a mixture thereof. In some embodiments, the one or more types of spore-forming bacteria comprise Clostridia Cluster IV, Clostridia Cluster XIVa or both. In some embodiments, the composition comprising one or more types of spore-forming bacteria comprises spore-forming microbes from a human intestine. In some embodiments, the composition comprising one or more types of spore-forming bacteria comprises spore-forming microbes from a healthy human colon. In some embodiments, at least, or at least about, 20%, 30%, 40%, 50%, 60%, 70%, 80%, 90%, 95%, 98% or more of the bacteria in the composition comprising one or more types of spore-forming bacteria are Clostridial species.

The composition comprising spore-forming bacteria can be in various forms, including but not limited to, a probiotic composition, a neutraceutical, a pharmaceutical composition, or a mixture thereof. In some embodiments, the composition is a probiotic composition.

In some embodiments, adjusting the composition of gut microbiota of the subject comprises administering the subject a composition comprising products derived from one or more types of spore-forming bacteria. Examples of products derived from bacteria include, but are not limited to, small molecules, polypeptides, lipids, enzymes, sugars, nucleic acids that are derived or produced from the bacteria, or any combination thereof.

Adjusting the level, for example blood level (e.g., serum and/or plasma level) and/or intestinal level (e.g., colonic level) of the serotonin-related metabolite in the subject can ameliorate various symptoms of the subject suffering from a serotonin-related disease. The symptoms can comprise abnormal GI motility, abnormal enteric motor and secretory reflexes, abnormal platelet aggregation, abnormal immune responses, abnormal bone development, abnormal cardiac function, abnormal hemostasis, abnormal mood, abnormal cognition, abnormal osteoblast differentiation, abnormal hepatic regeneration, abnormal erythropoiesis, abnormal intestinal immunity, abnormal neurodevelopment, or any combination thereof. As disclosed herein, amelioration is used in a broad sense to refer to at least a reduction in the magnitude of a parameter, e.g., symptom, associated with the pathological condition being treated. In some embodiments, the method can completely inhibited, e.g., prevented from happening, or stopped, e.g., terminated, such that the host no longer suffers from the pathological condition, or at least one or more of the symptoms that characterize the pathological condition. In some embodiments, the method can delay or slow disease progression, amelioration or palliation of the disease state, and/or remission (whether partial or total), whether detectable or undetectable. In some embodiments, adjusting the level of the serotonin-related metabolite improves GI motility of the subject.

After adjustment, the level of the serotonin-related metabolite in the subject can be, or be about, 50%, 60%, 70%, 80%, 90%, 95%, 98%, 99%, 100%, 101%, 102%, 105%, 110%, 120%, 130%, 140%, 150%, 200%, or a range between any two of these values, of the reference level of the metabolite in subjects having normal serotonin level (e.g., subjects that do not have, or are not at a risk of developing, serotonin-related diseases). In some embodiments, the level of the serotonin-related metabolite in the subject is at least, or is at least about, 80%, 85%, 90%, 95%, 98%, 99%, 100%, 101%, 102%, 105%, 110%, 120%, 130%, 150%, of the reference level of the metabolite in subjects having normal serotonin level. In some embodiments, the level of the serotonin-related metabolite in the subject is no more than, or is no more than about, 90%, 95%, 98%, 99%, 100%, 101%, 102%, 105%, or 110%, of the reference level of the metabolite in subjects having normal serotonin level. The level of the metabolite can be the level of the metabolite in circulation of the subject. For example, the level of the metabolite can be the level of the metabolite in blood or other body fluids (e.g., cerebrospinal fluid, pleural fluid, amniotic fluid, semen, or saliva) of the subject. In some embodiments, the level of the metabolite is the fecal level of the metabolite in the subject. In some embodiments, the level of the metabolite is the blood level of the metabolite in the subject. The blood level of the metabolite can be, for example, serum level or plasma level of the metabolite. In some embodiments, the level of the metabolite is the urine level of the metabolite in the subject.

The adjustment of the level of serotonin-related metabolite(s) in the subject can modulate serotonin biosynthesis in the subject in various extend. For example, the adjustment of the metabolite level may modulate (e.g., promote or reduce) the serotonin biosynthesis in the subject by, or by about, 5%, 10%, 20%, 30%, 40%, 50%, 60%, 70%, 80%, 90%, 100%, 101%, 102%, 105%, 110%, 120%, 130%, 140%, 150%, 200%, 250%, 300%, 400%, 500%, or a range between any two of these values. In some embodiments, the rate of serotonin biosynthesis after adjustment of the serotonin-related metabolite is, or is about, 1.05, 1.1, 1.2, 1.3, 1.4, 1.5, 2, or more times as compared to the rate of serotonin biosynthesis prior to the adjustment. In some embodiments, the adjustment of the metabolite level may reduce the serotonin biosynthesis in the subject by, or by about, 5%, 10%, 15%, 20%, 25%, 30%, 40%, 50%, 60%, 70%, 80%, 90%, or a range between any two of these values. In some embodiments, the rate of serotonin biosynthesis in the subject after adjustment of the serotonin-related metabolite is, or is about, 0.95, 0.9, 0.8, 0.7, 0.6, 0.5, 0.4, 0.3, 0.2, 0.1, 0.05, or less, of the rate of serotonin biosynthesis in the subject prior to the adjustment.

A variety of subjects are treatable. Generally, such subjects are mammals, where the term is used broadly to describe organisms which are within the class mammalia, including the orders carnivore (for example, dogs and cats), rodentia (for example, mice, guinea pigs and rats), and primates (for example, humans, chimpanzees and monkeys). In some preferred embodiments, the subjects are humans.

In the methods disclosed herein, the level of a metabolite in the subject can be determined by any conventional techniques known in the art, including but not limited to chromatography, liquid chromatography, size exclusion chromatography, high performance liquid chromatography (HPLC), gas chromatography, mass spectrometry, tandem mass spectrometry, matrix assisted laser desorption/ionization-time of flight (MALDI-TOF) mass spectrometry, electrospray ionization (ESI) mass spectrometry, surface-enhanced laser deorption/ionization-time of flight (SELDI-TOF) mass spectrometry, quadrupole-time of flight (Q-TOF) mass spectrometry, atmospheric pressure photoionization mass spectrometry (APPI-MS), Fourier transform mass spectrometry (FTMS), matrix-assisted laser desorption/ionization-Fourier transform-ion cyclotron resonance (MALDI-FT-ICR) mass spectrometry, secondary ion mass spectrometry (SIMS), radioimmunoassays, microfluidic chip-based assay, detection of fluorescence, detection of chemiluminescence, or a combination thereof.

### Methods for Treating Disorders Caused by Serotonin Deficiency

Methods for treating a disorder caused by serotonin deficiency are also provided herein. The methods include, in some embodiments, adjusting the composition of gut microbiota in a subject who is suffering from, or at a risk of developing a disorder caused by serotonin deficiency; and increasing the colonic, peripheral or blood level of serotonin in the subject. In some embodiments, the methods include administering to the subject a composition comprising one or more products derived from spore-forming bacteria. Examples of products derived from the spore-forming bacteria include, but are not limited to, small molecules, polypeptides, lipids, enzymes, sugars, nucleic acids that are produced from the bacteria, or any combination thereof.

The methods can further include determining the colonic or blood level of serotonin in the subject before the composition of gut microbiota in the subject is adjusted, after the composition of gut microbiota in the subject is adjusted, or both. For example, the level of serum, plasma and/or colonic serotonin of the subject can be determined.

As described herein, imbalance in serotonin level can cause abnormalities in many biological processes and functions. For example, serotonin deficiency can cause an abnormality in enteric motor and secretory reflexes, an abnormality in platelet aggregation, an abnormality in immune responses, an abnormality in bone development, an abnormality in cardiac function, an abnormality in gastrointestinal motility, an abnormality in hemostasis, an abnormality in mood, an abnormality in cognition, an abnormality in osteoblast differentiation, an abnormality in hepatic regeneration, an abnormality in erythropoiesis, an abnormality in intestinal immunity, an abnormality in neurodevelopment, or any combination thereof. Examples of diseases caused by serotonin deficiency include, but are not limited to, irritable bowel syndrome (IBS), inflammatory bowel disease (IBD), cardiovascular disease, osteoporosis, abnormal gastrointestinal motility, abnormal platelet aggregation, abnormal platelet activation, abnormal immune response, depression, anxiety, and a combination thereof. In some embodiments, the subject suffers from anxiety or depression. In some embodiments, the subject suffers from IBS. In some embodiments, the subject suffers from abnormal gastrointestinal motility.

As described herein, adjusting the composition of gut microbiota of the subject can comprise administering to the subject a composition comprising one or more types of spore-forming bacteria. The one or more types of spore-forming bacteria comprise, in some embodiments, Lactobacillales, Proteobacteria, Clostridia, or a mixture thereof. In some embodiments, the one or more types of spore-forming bacteria comprise Clostridia Cluster IV, Clostridia Cluster XIVa or both. The composition comprising one or more types of spore-forming bacteria can comprise dominantly Clostridia species. For example, at least 50%, 60%, 70%, 80%, 90% or 95% of the bacteria in the composition comprising one or more types of spore-forming bacteria can be Clostridia species. In some embodiments, the Clostridia species is Clostridia Cluster IV, Clostridia Cluster XIVa or both. In some embodiments, the composition comprising one or more types of spore-forming bacteria comprises spore-forming microbes from a human intestine (e.g., a healthy human colon or small intestine). In some embodiments, the composition comprising one or more types of spore-forming bacteria is a probiotic composition, a neutraceutical, a pharmaceutical composition, or a mixture thereof.

In some embodiments, the method for treating a disorder caused by serotonin deficiency comprises adjusting the level of one or more serotonin-related metabolites in the subject in need of treatment, wherein the adjustment of the metabolite level increases the colonic, peripheral or blood level of serotonin in the subject. In some embodiments, the method comprises adjusting the level of one or more metabolites listed in Table 1. In some embodiments, the serotonin-related metabolite is deoxycholate, α-tocopherol, tyramine, p-aminobenzoate, or any combination thereof.

In the methods disclosed herein, the amount of substance (for example, bacteria (e.g., spore-forming bacteria), bacterial product, serotonin-related metabolite, enzyme, intermediate, substrate, or a combination thereof) for administering to the subject can be determined according to various parameters such as the age, body weight, response of the subject, condition of the subject to be treated; the type and severity of the pathological conditions; the form of the composition in which the substance is included; the route of administration; and the required regimen. The severity of the condition may, for example, be evaluated, in part, by standard prognostic evaluation methods. A program comparable to that discussed above may be used in veterinary medicine. For example, the amount of the substance can be titrated to determine the effective amount for administering to the subject in need of treatment. One of skill in the art would appreciate that the attending physician would know how to and when to terminate, interrupt or adjust administration of the substance due to toxicity or organ dysfunctions. Conversely, the attending physician would also know to adjust treatment to higher levels if the clinical response were not adequate (precluding toxicity).

### EXAMPLES

Some aspects of the embodiments discussed above are disclosed in further detail in the following examples, which are not in any way intended to limit the scope of the present disclosure.

### Experimental Materials and Methods

The following experimental materials and methods were used for Examples 1-5 described below.

### Animals

SPF C57B1/6J mice and SPF Slc6a4 knockout (KO) mice (Jackson Laboratories) were bred in Caltech's Broad Animal Facility. GF C57B1/6J mice (rederived from SPF C57B1/6J mice from Jackson Laboratories), GF Swiss Webster mice, GF Rag1 KO, B. fragilis monoassociated and SFB monoassociated mice were bred in Caltech's Gnotobiotic Animal Facility. GF Slc6a4 KO mice were generated by C-section rederivation, cross-fostering to GF Swiss Webster mice (Taconic Farms) and bred as an independent GF line in Caltech's Gnotobiotic Animal Facility. All animal experiments were approved by the Caltech IACUC.

### Human Biopsy Sample and Colonization of GF Mice

Archived, de-identified clinical samples of colonic microbiota were provided by Eugene Chang at the University of Chicago and handled as described previously in Ma et al., PNAS USA 111:9768-9773 (2014). Briefly, a sample of mucosal brush and luminal aspirate from the colon of a healthy human subject was placed on ice, transferred into an anaerobic chamber immediately after collection and homogenized in grants buffered saline solution (GBSS) supplemented with 5% DMSO by vortexing for 5 minutes (min). Aliquots of the samples were flash frozen with liquid nitrogen and preserved at −80° C. 100 μl of the suspension was used to gavage founder GF mice, housed in a designated gnotobiotic isolator.

### Microbiota Conventionalization

Fecal samples were freshly collected from adult SPF C57B1/6J mice and homogenized in prereduced PBS at 1 mL per pellet. 100 μl of the settled suspension was administered by oral gavage to postnatal day (P) 21 and P42 GF mice. For conventionalization at P0, GF mothers were gavaged with 100 μl of the SPF fecal suspension, and the mother and litter were transferred into a dirty cage, previously housed for 1 week with adult SPF C57B1/6J mice. For mock treatment, mice were gavaged with pre-reduced PBS.

### Antibiotic Treatment

P21 and P42 SPF mice were gavaged with a solution of vancomycin (50 mg/kg), neomycin (100 mg/kg), metronidazole (100 mg/kg) and amphotericin-B (1 mg/kg) every 12 hours daily until P56, according to methods described in (Reikvam et al., 2011). Ampicillin (1 mg/mL) was provided ad libitum in drinking water. For antibiotic treatment at P0, drinking water was supplemented with ampicillin (1 mg/mL), vancomycin (500 mg/mL) and neomycin (1 mg/mL) until P21, and from P21-P56, mice were gavaged with antibiotics as described above. For mock treatment, P42 mice were gavaged with unsupplemented drinking water every 12 hours daily until P56.

### Bacterial Treatment

Frozen fecal samples from Sp- and ASF-colonized mice were generously supplied by the laboratory of Cathryn Nagler (University of Chicago). Fecal samples were suspended at 50 mg/ml in pre-reduced PBS, and 100 μl was orally gavaged into adult C57B1/6J GF mice. These “founder” mice were housed separately in dedicated gnotobiotic isolators and served as repositories for fecal samples used to colonize experimental mice. For generation of “founder” mice colonized with human spore-forming bacteria, fecal pellets were collected from humanized mice, described above, and suspended in a 10× volume of pre-reduced PBS in an anaerobic chamber. Chloroform was added to 3% (vol/vol), the sample was shaken vigorously and incubated at 37° C. for 1 hr. Chloroform was removed by percolation with CO2 from a compressed cylinder, and 200 μl suspension was orally gavaged into adult C57B1/6J GF mice housed in designated gnotobiotic isolators.

Fecal samples were collected from founder mice and immediately frozen at −80° C. for later Sp or ASF colonization. Experimental GF or antibiotic-treated mice were colonized on P42 by oral gavage of 100 μl of 50 mg/ml fecal suspension in pre-reduced PBS. For mock treatment, mice were gavaged with pre-reduced PBS. For the Bacteroides (Bd) consortium, feces from adult SPF Swiss Webster mice was suspended at 100 mg/ml in BHI media and serially plated on Bacteroides Bile Esculin (BBE) agar (BD Biosciences). 100 μl of a 1000 cfu/ml suspension in PBS was used for colonization of P42 GF mice. Colony PCR and sequencing indicates that among the most abundant species in the Bd consortium are B. thetaiotaomicron, B. acidifaciens, B. vulgatus and B. uniformis.

### PCPA Treatment

At 2 weeks post-bacterial treatment, mice were anesthetized with isoflurane, and PCPA (90 mg/kg) (Liu et al., 2008) was administered intrarectally every 12 hours for 3 days using a sterile 3.5 Fr silicone catheter inserted 4 cm into the rectum. Mice were suspended by tail for 30 seconds(s) before return to the home cage. For mock treatment, mice were anesthetized and intrarectally injected with sterile water as vehicle.

### Serotonin Measurements

Blood samples were collected by cardiac puncture and spun through SST vacutainers (Becton Dickinson) for serum separation or PST lithium hepararin vacutainers (Becton Dickinson, Franklin Lakes, NJ) for plasma separation. The entire length of the colon or 1 cm regions of the distal, medial and proximal colon of the small intestine were washed in PBS, flushed with PBS to remove luminal contents, and sonicated on ice in 10 s intervals at 20 mV in ELISA standard buffer supplemented with ascorbic acid (Eagle Biosciences, Nashua, NH). Serotonin levels were detected in sera and supernatant of tissue homogenates by ELISA according to the manufacturer's instructions (Eagle Biosciences, Nashua, NH). Readings from tissue samples were normalized to total protein content as detected by BCA assay (Thermo Pierce). Data compiled across multiple experiments are expressed as 5-HT concentrations normalized to SPF controls within each experiment.

Intestinal qRT-PCR

The entire length of the mouse colon, or 1 cm regions of the distal, medial and proximal of the mouse small intestine were washed in PBS, flushed with PBS to remove luminal contents, and homogenized in ice-cold Trizol for RNA isolation using the RNeasy Mini Kit with on-column genomic DNA-digest (Qiagen) and cDNA synthesis using iScript (Biorad). qRT-PCR was performed on an ABI 7900 thermocycler using SYBR green master mix with Rox passive reference dye (Roche) and validated primer sets obtained from Primerbank (Harvard).

### RIN14B In Vitro Culture Experiments

RIN14B cells (ATCC) were seeded at 105 cells/cm² and cultured for 3 days in RPMI 1640 supplemented with 10% FBS, 100 U/mL penicillin and 100 μg/mL streptomycin according to methods previously described in Nozawa et al., PNAS USA 106:3408-3413 (2009). Total colonic luminal contents were collected from adult SPF, GF and GF mice colonized with spore-forming bacteria, suspended at 120 μl/mg in HBSS supplemented with 0.1% BSA and 2 uM fluoxetine, and centrifuged at 12,000×g for 10 min. Supernatants were passed through 0.2 um pore syringe filters. Cultured RIN14B cells were incubated with colonic luminal filtrate at 125 μl/cm² for 1 hour (hr) at 37° C. Positive controls were incubated with 15 uM ionomycin in vehicle (HBSS). After incubation, supernatant was collected, centrifuged at 6000×g for 5 min to pellet any residual cells, and frozen for downstream 5-HT assays. Remaining adherent RIN14B cells were lysed in Trizol for downstream RNA isolation, cDNA synthesis and qRT-PCR as described above. For experiments with colonic luminal contents, starting 5-HT levels in filtrate were subtracted from post-assay 5-HT levels, and this difference is reported as “5-HT released”.

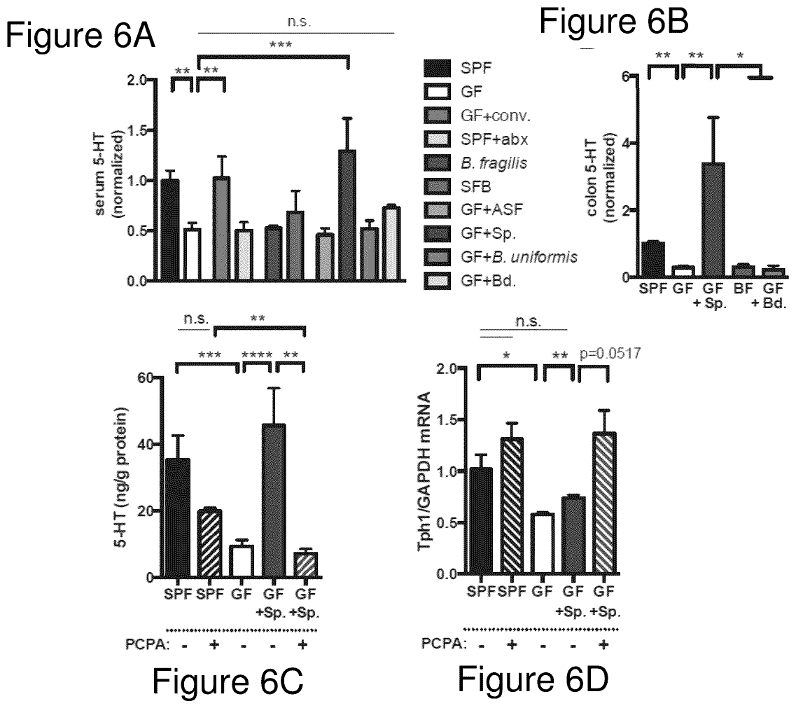

For metabolite sufficiency assays, cells were incubated with biochemicals in HBSS or 1% DMSO in HBSS at the indicated concentrations. Pilot experiments were conducted to test the ability of physiologically relevant concentrations (as identified in existing scientific literature) of acetate, α-tocopherol, arabinose, azelate, butyrate, cholate, deoxychoate, ferulate, GABA, glycerol, N-methyl proline, oleanolate, p-aminobenzoate (PABA), propionate, taurine, and tyramine to induce 5-HT in RIN14B cultures. 5-HT concentrations were normalized to levels detected in the appropriate RIN14B+vehicle (HBSS or 1% DMSO in HBSS) control. For biochemicals that raised 5-HT levels in culture, additional pilot experiments were conducted to determine the lowest concentrations possible for elevating 5-HT in vitro. These concentrations were further tested in triplicate to generate the data presented in FIG. 6D.

### Intestinal Histology and Immunofluorescence Staining

Mouse colon was cut into distal, medial and proximal sections, and 1 cm regions of the distal, medial and proximal small intestine were fixed in Bouin's solution (Sigma Aldrich) overnight at 4° C., washed and stored in 70% ethanol. Intestinal samples were then paraffin-embedded and cut into 10 um longitudinal sections by Pacific Pathology, Inc (San Diego, CA). Sections were stained using standard procedures. Briefly, slides were deparaffinized, and antigen retrieval was conducted for 1 hr in a 95° C. water bath in 10 mM sodium citrate, pH 6.0 or DAKO solution (Agilent Technologies). Slides were washed, blocked in 5% normal serum or bovine serum albumin (Sigma Aldrich), and stained using the primary antibodies, rabbit anti-mouse CgA (1:500; Abcam), rat anti-mouse 5-HT (1:50; Abcam), rabbit anti-mouse c-fos (1:100; Abcam), goat anti-mouse calretinin (1:1500; Millipore), rabbit anti-5HT4 (1:3000; Abcam), and secondary antibodies conjugated to Alexa fluor 488 or 594 (Molecular Probes). Slides were mounted in Vectashield (Vector Labs), and 3-15 images were taken per slide at 20× or 40× magnification along transections of the intestinal crypts for each biological replicate (EVOS FL System; Life Technologies). Monochrome images were artificially colored, background corrected and merged using Photoshop CS5 (Adobe). For 5-HT and CgA staining, numbers of positively-stained puncta were scored blindly, normalized to total area of intestinal mucosa using ImageJ software (NIH) (Schneider et al., Nature Methods 9:671-675 (2012)), and then averaged across biological replicates. For calretinin, cfos and 5HT4 staining, fluorescence intensity for individual stains was quantified and normalized to total area of intestinal submucosa and muscularis externa using ImageJ software. Colocalization was measured and analyzed using the Coloc2 plug-in for Fiji software described in Schindelin et al., Nature Methods 9:676-682 (2012). Representative images are presented in the figures, where Alexa fluor 594 staining is replaced with magenta.

### GI Transit Assay

Mice were orally gavaged with 200 μl sterile solution of 6% carmine red (Sigma Aldrich) and 0.5% methylcellulose (Sigma Aldrich) in water, and placed in a new cage with no bedding (Li et al., J. Neurosci. 31:8998-9009 (2011)). Starting at 120 minutes post-gavage, mice were monitored every 10 minutes for production of a red fecal pellet. GI transit time was recorded as the total number of minutes elapsed (rounded to the nearest 10 minutes) before production of a red fecal pellet. For mice treated intrarectally with PCPA or metabolites, GI transit assay was conducted 1 hour after the third injection.

### Platelet Activation and Aggregation Assays

Blood samples were collected by cardiac puncture, diluted with a 2× volume of HEPES medium (132 mM NaCl, 6 mM KCl, 1 mM MgSO₄, 1.2 mM KH₂PO₄, 20 mM HEPES, 5 mM glucose; pH 7.4) and centrifuged through PST lithium hepararin vacutainers (Becton Dickinson). Expression of platelet activation markers was measured by flow cytometry (Nieswandt et al., Methods Mol. Biol. 272:255-268 (2004); Ziu et al., J. Mol. Cell. Cardiol. 52:1112-1121 (2012)). Briefly, PRP samples were supplemented with 1 mM CaCl2, and 1×106 platelets were stimulated with 10 μg/ml type-1 HORM collagen (Chronolog), and stained with anti-JON/APE, anti-P-selectin-FITC (Emfret Analytics), anti-CD63-PE (Biologend), anti-CD41-FITC (BD Biosciences) and anti-CD9-APC (Abcam) for 15 min at room temperature. Samples were then washed in PBS, fixed with 0.5% formaldehyde and analyzed using a FACS Calibur flow cytometer (BD Biosciences). Platelet aggregation assays were conducted according to methods described in (De Cuyper et al., Blood 121:070-e80 (2013)). Briefly, 4×10⁶ platelets were stained separately with CD9-APC or CD9-PE (Abcam) for 15 minutes at room temperature and then washed with HEPES medium. Labeled platelets were mixed 1:1 and incubated for 15 minutes at 37° C., with shaking at 600 rpm. Platelets were then stimulated with 10 μg/ml type-1 collagen for 2 min and fixed in 0.5% formaldehyde for flow cytometry. Remaining unstained PRP was treated with collagen as described above, and then used to generate PRP smears. Slides were stained with Wright Stain (Camco) according to standard procedures. Platelets were imaged at 200× magnification, and 9 images were taken across each PRP smear, processed using ImageJ software (intensity threshold: 172, size threshold: 500) (Schneider et al., 2012), totaled for each biological replicate, and then averaged across biological replicates. Comprehensive complete blood counts were conducted by Idexx Laboratories using the ProCyte Dx Hematology Analyzer.

### Tail Bleed Assay

Mice were anesthetized with isoflurane and the distal 6 mm portion of the tail was transected using a fresh razor blade. The tail was placed immediately at a 2 cm depth into a 50 mL conical tube containing saline pre-warmed to 37° C. (Liu et al., World J. Exp. Med. 2:30-36 (2012)). Time to bleeding cessation was recorded, with continued recording if re-bleeding occurred within 15 seconds of initial cessation and a maximum total bleed time of 5 minutes. Mice were sacrificed by CO2 immediately after the assay.

### Metabolomics Screening

Fecal samples were collected from adult mice at 2 weeks post-bacterial treatment, and immediately snap frozen in liquid nitrogen. Each sample consisted of 3-4 fecal pellets freshly collected between 9-11 am from mice of the same treatment group co-housed in a single cage. Samples were prepared using the automated MicroLab STAR system (Hamilton Company) and analyzed on GC/MS, LC/MS and LC/MS/MS platforms by Metabolon, Inc. Protein fractions were removed by serial extractions with organic aqueous solvents, concentrated using a TurboVap system (Zymark) and vacuum dried. For LC/MS and LC-MS/MS, samples were reconstituted in acidic or basic LC-compatible solvents containing >11 injection standards and run on a Waters ACQUITY UPLC and Thermo-Finnigan LTQ mass spectrometer, with a linear ion-trap front-end and a Fourier transform ion cyclotron resonance mass spectrometer back-end. For GC/MS, samples were derivatized under dried nitrogen using bistrimethyl-silyl-trifluoroacetamide and analyzed on a Thermo-Finnigan Trace DSQ fast-scanning single-quadrupole mass spectrometer using electron impact ionization. Chemical entities were identified by comparison to metabolomic library entries of purified standards. Following log transformation and imputation with minimum observed values for each compound, data were analyzed using Welch's two-sample t-test.

### Metabolite In Vivo Injection Experiments

Adult GF C57B1/6 mice were anesthetized with isoflurane, and metabolites were injected intrarectally (α-tocopherol: 2.25 mg/kg, deoxycholate: 125 mg/kg, oleanolate: 0.457 mg/kg) using a sterile 3.5 Fr silicone catheter (Solomon Scientific). Concentrations were based on levels reported in Sayin et al., Cell Metab. 17:225-235 (2013), Alemi et al., Gastroenterology 144:145-154 (2013) and (Zhao et al., Journal of Agricultural and Food Chemistry 58:4844-4852 (2010). Mice were suspended by tail for 30 s before return to the home cage. For mock treatment, GF mice were anesthetized and intrarectally injected with vehicle. For experiments evaluating physiological effects of metabolite administration, adult GF mice were injected every 12 hours for 3 days. GI motility assays were initiated at 1 hour after the third injection (day 2). For 5-HT measurements and platelet assays, mice were sacrificed at 1 hour after the final injection. For pilot time course experiments, adult GF Swiss Webster mice were injected once, as described above, and sacrificed at the indicated time points post-injection. Use of the Swiss Webster strain was based on availability and our validation that microbiota effects on colonic and blood 5-HT levels are similarly seen in both the Swiss Webster and C57B1/6 mouse strains.

16S rRNA Gene Sequencing and Analysis

This experiment evaluates microbes recovered from Sp and hSp-colonized mice, and may not reflect the full microbial diversity within the initial inoculum. Fecal samples were collected at two weeks after orally gavaging GF mice with Sp or hSp. Fecal pellets were bead-beaten in ASL buffer (Qiagen) with lysing matrix B (MP Biomedicals 6911-500) in a Mini-Beadbeater-16 (BioSpec Products, Inc.) for 1 min. Bacterial genomic DNA was extracted from mouse fecal pellets using the QIAamp DNA Stool Mini Kit (Qiagen) with InhibitEX tables. The library was generated according to methods adapted from Caporaso et al., PNAS USA 108 (Suppl 1): 4516-4522 (2011. The V4 regions of the 16S rRNA gene were PCR amplified using individually barcoded universal primers and 30 ng of the extracted genomic DNA. The PCR reaction was set up in triplicate, and the PCR product was purified by Agencourt AmPure XP beads (Beckman Coulter Inc, A63881) followed by Qiaquick PCR purification kit (Qiagen). The purified PCR product was pooled in equal molar quantified by the Kapa library quantification kit (Kapa Biosystems, KK4824) and sequenced at UCLA's GenoSeq Core Facility using the Illumina MiSeq platform and 2×250 bp reagent kit. Operational taxonomic units (OTUs) were chosen de novo with UPARSE pipeline described in Edgar, Nat. Methods 10:996-998 (2013). Taxonomy assignment and rarefaction were performed using QIIME1.8.0 (Caporaso et al., Nat. Methods 7:335-336 (2010)).

Phylogenetic trees were built using PhyML (Guindon et al., Syst. Biol. 59:307-321 (2010)) (General Time Reversible model, subtree pruning and regrafting method, with ten random start trees) and visualized using iTOL (Letunic and Bork, Bioinformatics 23:127-128 (2007)). The 32 most abundant OTUs in Sp and hSp were included after excluding OTUs that were only present in more than 50% of biological replicates from sequenced fecal samples. Sequenced genomes from JGI's Integrated Microbial Genomes database (Markowitz et al., Nucleic Acids Res. 40: D115-D122 (2012)) were searched for enzymes of interest (EC: 1.4.3.4 monoamine oxidase, EC: 2.1.1.95 tocopherol o-methyltransferase, EC: 1.2.1.68 coniferyl-aldehyde dehydrogenase, EC: 4.1.1.25 tyrosine decarboxylase). Hits phylogenetically related to the OTUs from Sp or to sequenced genomes from B. fragilis, B. uniformis, B. vulgatus, B. thetaiotaomicron, B. acidifaciens and SFB were included. Bacteria with 7α-dehydroxylation activity were identified from previous reports (Hirano et al., Appl. Environ. Microbiol. 41:737-745 (1981); Kitahara et al., Int. J. Syst. Evol. Microbiol. 50:971-978 (2000); Kitahara et al. Int. J. Syst. Evol. Microbiol. 51:39-44 (2001)).

### Statistical Analysis

Statistical analysis was performed using Prism software (Graphpad). Data were assessed for normal distribution and plotted in the figures as mean±SEM. Differences between two treatment groups were assessed using two-tailed, unpaired Student t test with Welch's correction. Differences among >2 groups were assessed using one-way ANOVA with Bonferroni post hoc test. Two-way ANOVA with Bonferroni post hoc test was used to assess treatment effects in PCPA experiments involving >2 experimental groups (e.g. SPF, GF, Sp). Welch's two-sample t-test was used for analysis of metabolomic data. Significant differences emerging from the above tests are indicated in the figures by *p<0.05, **p<0.01, ***p<0.001, ****p<0.0001 Notable near-significant differences (0.5<p<0.1) are indicated in the figures. Notable nonsignificant (and non-near significant) differences are indicated in the figures by “n.s.”

### Trp/5-HTP Supplementation Experiment

Water was supplemented with Trp, 5-HTP or 5-HT at 1.5 mg/ml (based on calculations from Abdala-Valencia et al., Am. J. Physiol. Lung Cell Mol. Physiol. 303: L642-L660 (2012) and provided ad libitum to mice for 2 weeks. Amount of water consumed and mouse weight was measured on days 3, 7, 10 and 14 of treatment. Mice were sacrificed one day after treatment for 5-HT assays.

### Slc6a4 Mouse Antibiotic Treatment and Sp Colonization

Adult Slc6a4 mice were gavaged with a solution of vancomycin (50 mg/kg), neomycin (100 mg/kg), metronidazole (100 mg/kg) and amphotericin-B (1 mg/kg) every 12 hours daily for 2 weeks, according to methods described in (Reikvam et al., 2011). Ampicillin (1 mg/mL) was provided ad libitum in drinking water. For Sp colonization, mice were orally gavaged 2 days after the final antibiotic treatment with 100 μl of 50 mg/mL fecal suspension in pre-reduced PBS. For mock treatment, mice were gavaged with pre-reduced PBS. Mice were then tested in 5-HT-related assays 2 weeks after oral gavage.

### Example 1

### The Gut Microbiota Modulates Host Peripheral Serotonin Levels

This example demonstrates the key role of gut microbiota in elevating the levels of 5-HT.

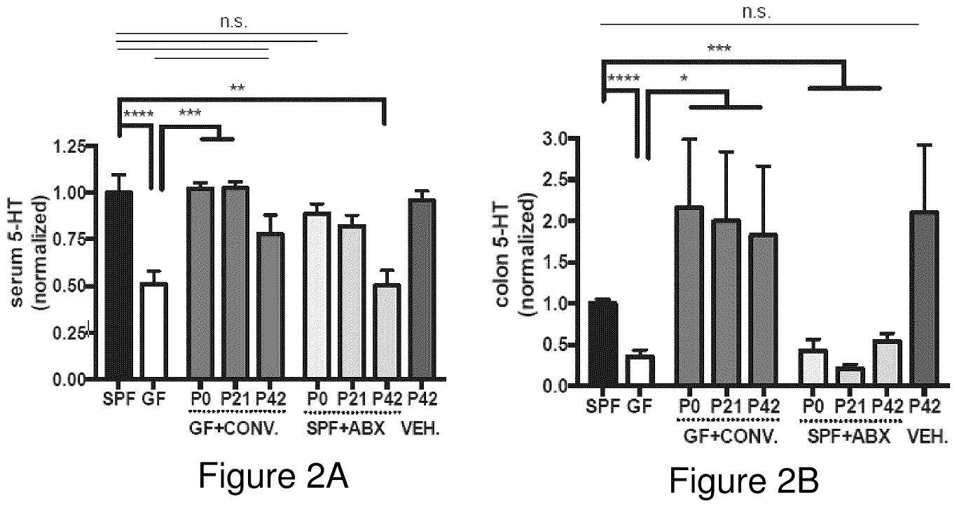

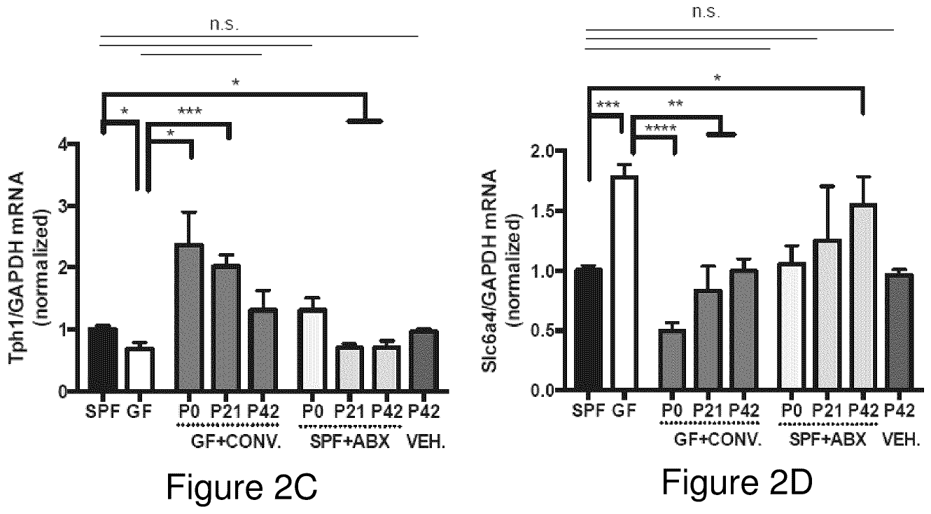

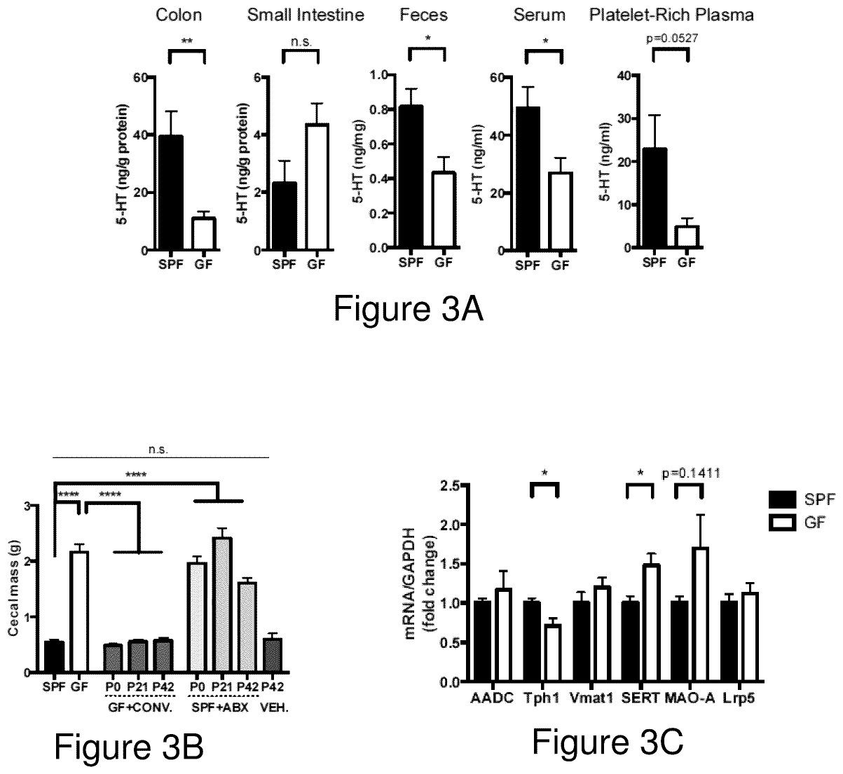

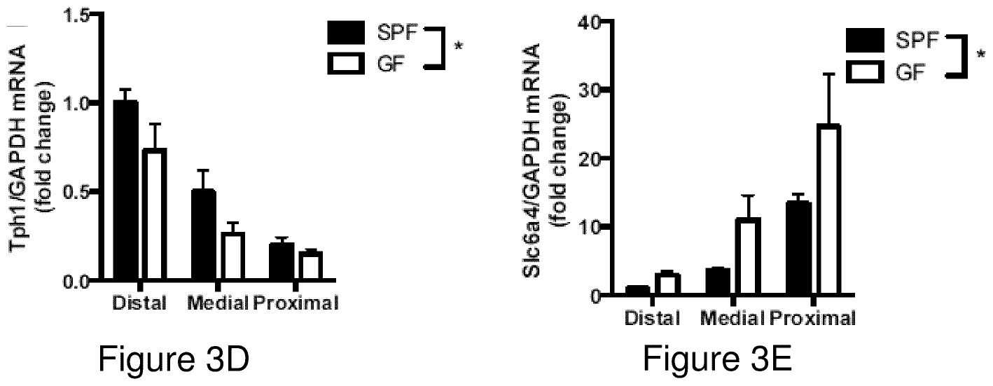

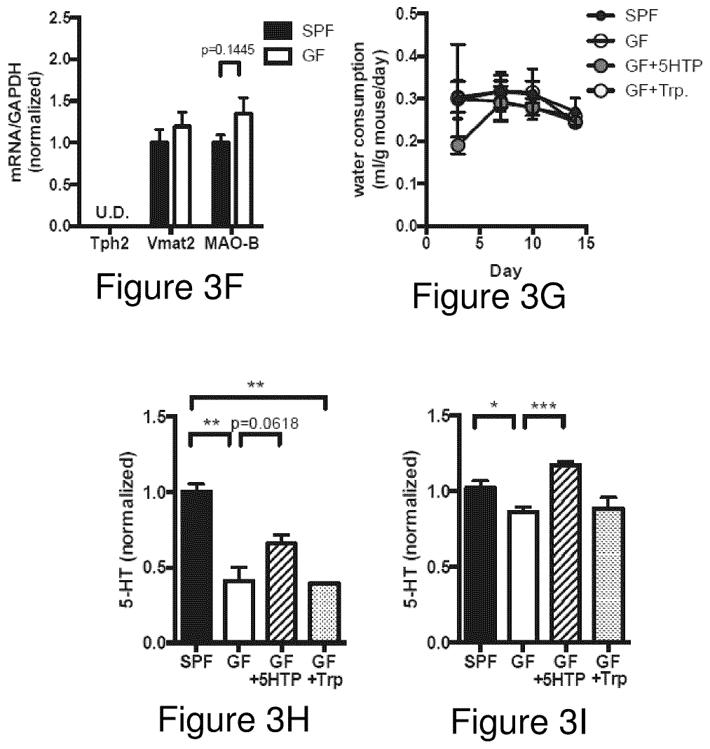

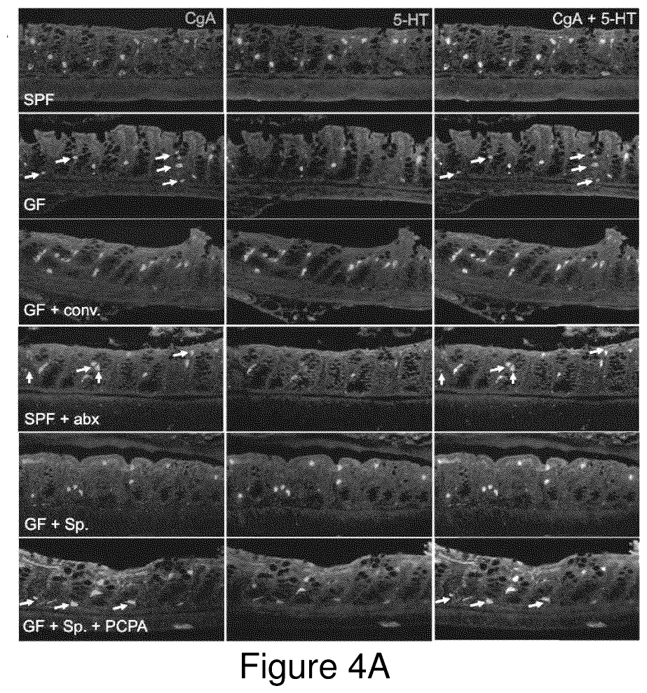

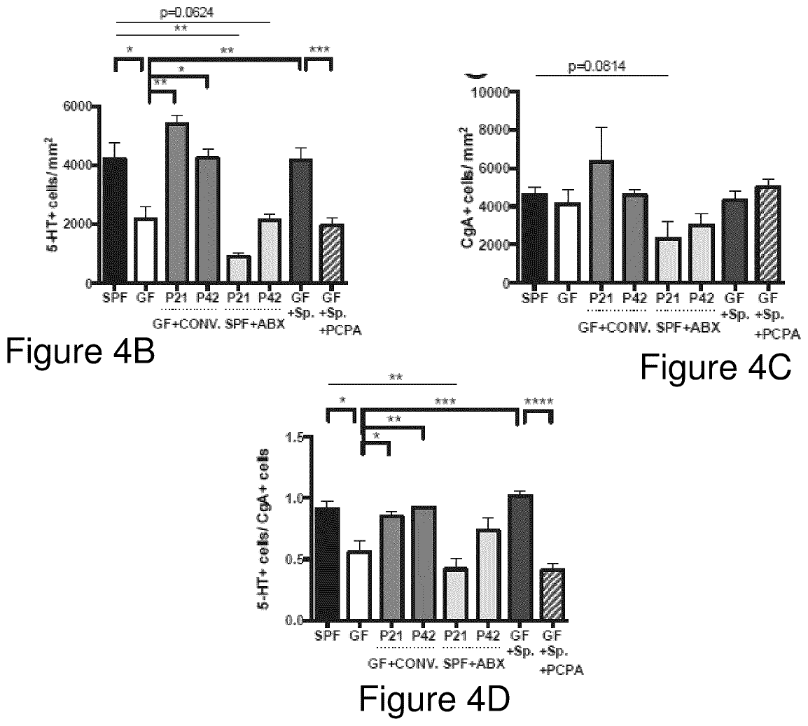

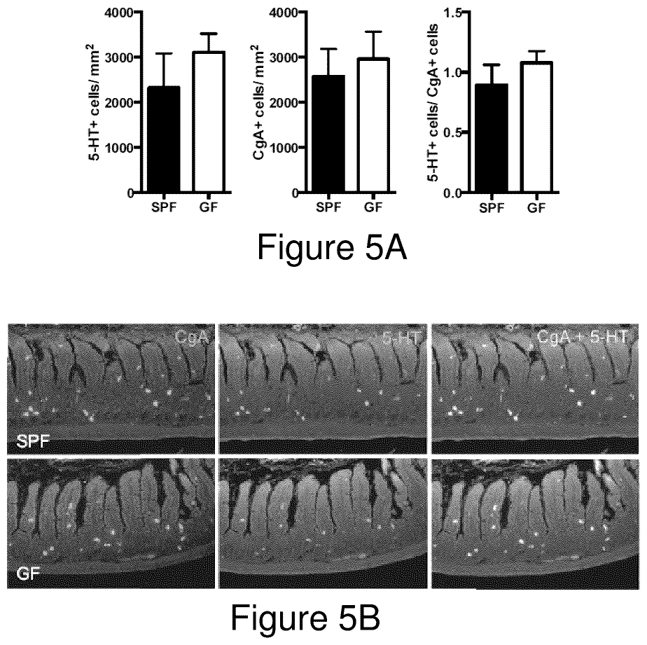

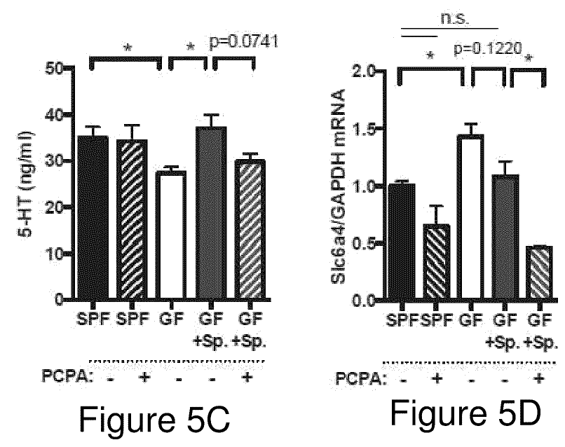

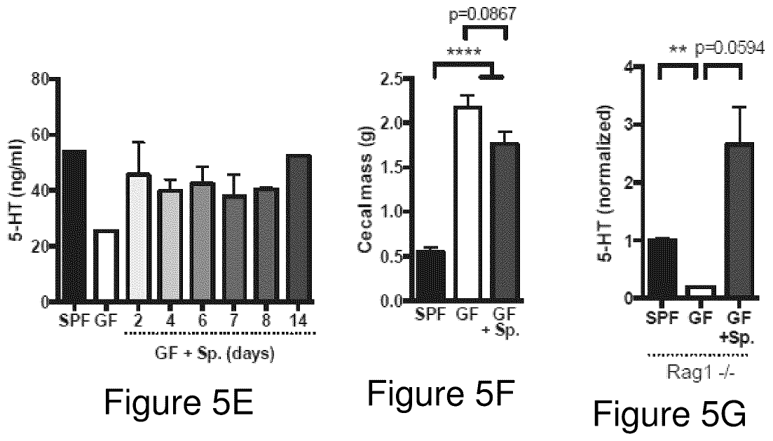

Adult GF mice exhibit dramatic decreases in levels of serum (FIG. 2A) and plasma (FIG. 3A) 5-HT compared to SPF controls, but the cellular sources of this disruption are undefined. Consistent with the previous understanding that much of the body's 5-HT derives from the GI tract, it was found that GF mice exhibit significantly decreased levels of colonic and fecal 5-HT compared to SPF controls (see e.g., FIGS. 2B and 3A). This deficit in 5-HT is observed broadly across the distal, medial and proximal colon (FIG. 3D), but not in the small intestine (FIGS. 3A, 5A and 5B), suggesting a specific role for the microbiota in regulating colonic 5-HT. Decreased levels of 5-HT are localized to colonic chromogranin A-positive (CgA+) enterochromaffin cells (ECs) (FIGS. 4A-4D), and not to small intestinal ECs (FIGS. 5A and 5B). Low 5-HT signal is seen in both GF and SPF colonic mast cells and enteric neurons (FIG. 4A), which are reported to be minor producers of 5-HT. That there is no difference between adult GF and SPF mice in the abundance of CgA+enteroendocrine cells (EECs) (FIG. 4C) suggests that decreases in colon 5-HT result from abnormal 5-HT metabolism rather than impaired development of EECs.

To identify the specific steps of 5-HT metabolism that are affected by the microbiota, key enzymes and intermediates of the 5-HT pathway were assessed in colons from GF vs. SPF mice. It was found that GF colons exhibit decreased expression of TPH1 (FIGS. 2C and 3D), the rate-limiting enzyme for 5-HT biosynthesis in ECs, but no difference in expression of enzymes involved in 5-HT packaging, release and catabolism (FIG. 3C). GF mice also display elevated colonic expression of the 5-HT transporter SLC6A4 (FIGS. 2D and 3E), synthesized broadly by enterocytes to enable 5-HT uptake (Wade et al., J. Neurosci. 16:2352-2364 (1996)). Without being bound by any particular theory, it is believe that this could reflect a compensatory response to deficient 5-HT synthesis by host ECs, based on the finding that chemical Tph inhibition modulates SLC6A4 expression (FIGS. 5C and 5D). There is no difference between GF and SPF mice in colonic expression of neural-specific isoforms of 5-HT enzymes (FIG. 3F), consistent with immuno-histochemical data showing no apparent difference in 5-HT-specific staining in enteric neurons (FIGS. 4A-4D). Despite deficient levels of colon, fecal and serum 5-HT (see e.g., FIGS. 2A, 2B and 3A) GF mice exhibit dramatically increased levels of the Tph substrate, tryptophan (Trp), in both serum (Sjogren et al., J. Bone Miner Res. 27:1357-1367 (2012); Wikoff et al., PNAS USA 106:3698-3703 (2009)) and feces, suggesting that primary disruptions in host TPH1 expression result in Trp accumulation. Oral supplementation of GF mice with the Tph product, 5-hydroxytryptophan (5-HTP), sufficiently ameliorates deficits in colon and serum 5-HT, whereas supplementation with the Tph substrate Trp has no restorative effect (FIGS. 3G, 3H and 3I). Collectively, these data support the notion that the microbiota promotes 5-HT biosynthesis by elevating TPH1 expression in colonic ECs.

To confirm that deficient 5-HT levels in GF mice are microbiota-dependent, and further determine whether effects are age-dependent, GF mice were conventionalized with an SPF microbiota at birth (postnatal day (P0), weaning (P21), or early adulthood (P42) and then evaluated at P56 for levels of 5-HT and expression of 5-HT-related genes. GF mice conventionalized at each age with an SPF microbiota exhibit restored serum (FIG. 2A) and colon (FIG. 2B) 5-HT levels, with more pronounced effects seen at earlier ages of colonization. Host colonic expression of TPH1 and SLC6A4 is similarly corrected by postnatal conventionalization of GF mice (FIGS. 2C and 2D), with more substantial changes from P0 conventionalization. Increases in intestinal 5-HT are localized to colonic ECs (FIGS. 4A-4D). Restoration of cecal weight is also observed with conventionalization at P0, P21 and P42 (FIG. 3B). These findings indicate that postnatal reconstitution of the gut microbiota can correct the 5-HT deficiency seen in GF mice and further suggest that indigenous microbes exert a continuous effect on 5-HT synthesis by modulating EC function. Overall, this demonstrates that microbiota-mediated elevation of host 5-HT is post-natally inducible, persistent from the time of conventionalization and not dependent on the timing of host development.

To assess the reversibility of microbial effects on host 5-HT metabolism, the gut microbiota in SPF mice were depleted via bi-daily broad-spectrum antibiotic treatment beginning on P0, P21 or P42 and until P56. Treatment of P42 SPF mice with a cocktail of ampicillin, vancomycin, neomycin and metronidazole (Reikvam et al., PLOS ONE 6: e17996 (2011)) sufficiently recapitulates GF-associated deficits in serum and colon 5-HT and alterations in host colonic TPH1 and SLC6A4 expression (FIGS. 2A-2D and 4A-4D). Interestingly, P0 and P21 antibiotic treatment also induces GF-related deficits in colonic 5-HT, but the effects on serum 5-HT are more pronounced when administered at P42, compared to P0 and P21 (FIGS. 2A-2D), indicting potential confounding effects of early life or prolonged antibiotic treatment on microbiota-mediated modulation of peripheral 5-HT. Notably, antibiotics can elicit several direct effects on host cells, which may underlie differences between antibiotic treatment from birth and germ-free status. That P42 antibiotic treatment of SPF mice results in 5-HT phenotypes analogous to those seen in GF mice demonstrates that microbiota effects on host 5-HT levels can be abrogated post-natally and further supports the plasticity of 5-HT modulation by indigenous gut microbes.

Altogether, these data indicate that the gut microbiota plays a key role in raising levels of colon and serum 5-HT, by for example promoting 5-HT in colonic ECs in an inducible and reversible manner.

### Example 2

### Indigenous Spore-Forming Microbes Promote Host Serotonin Biosynthesis

This example demonstrates that indigenous spore-forming microbes promote host serotonin biosynthesis, for example by promoting tryptophan hydroxylase 1-mediated serotonin biosynthesis by colonic enterochromaffin cells.

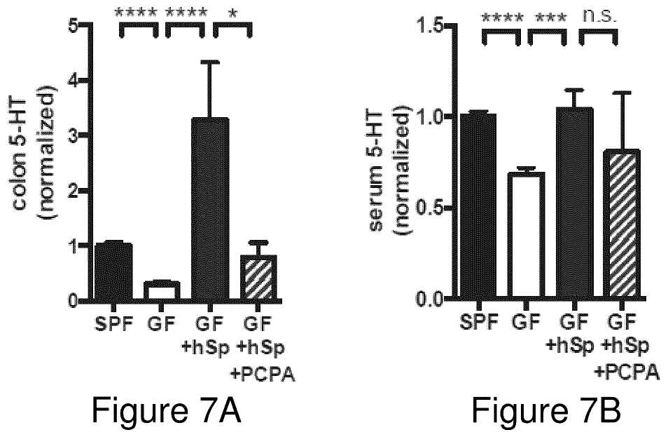

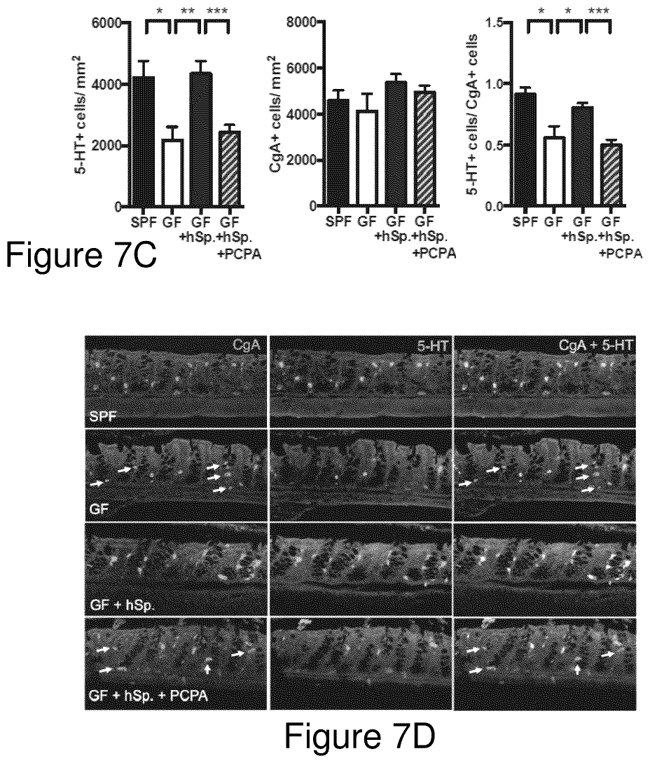

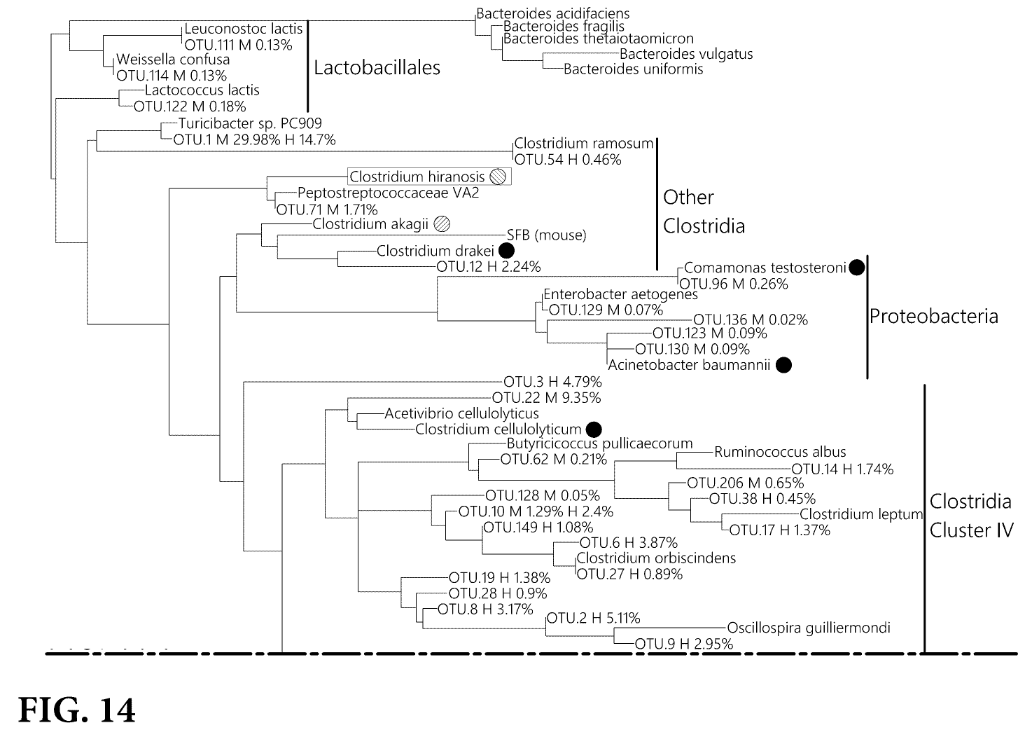

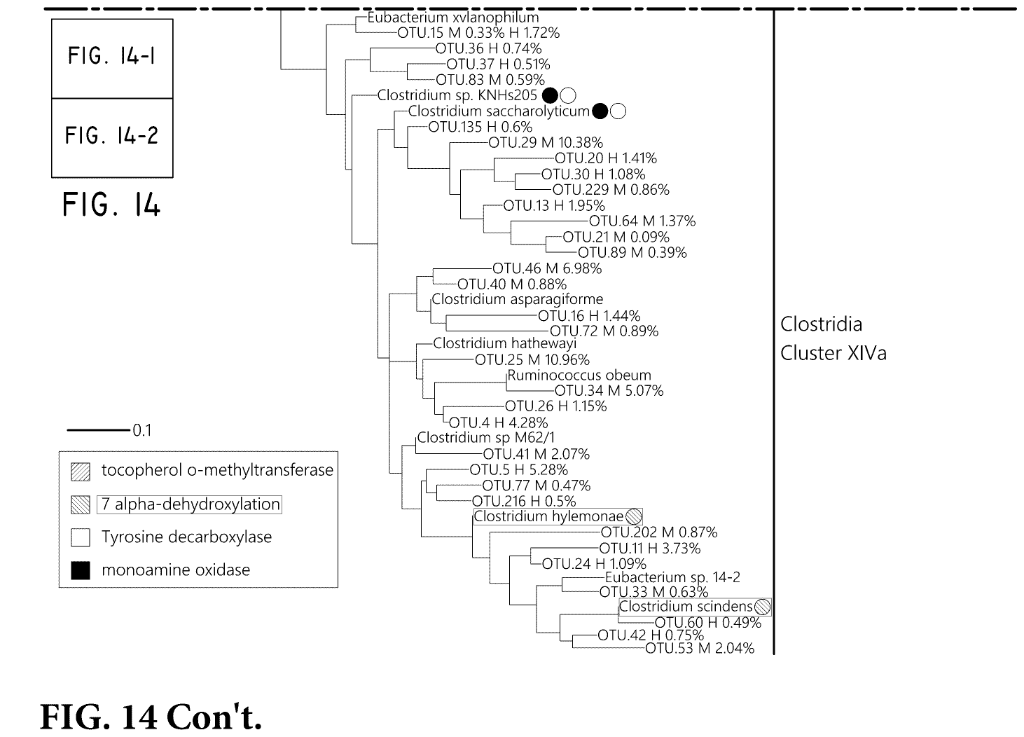

The mammalian colon harbors a far greater abundance and diversity of microbes than does the small intestine. In light of our finding that 5-HT levels are decreased in colons but not small intestines of GF mice compared to SPF controls, it was hypothesized that specific subsets of gut microbes are responsible for affecting host 5-HT pathways. Mice monocolonized with the human symbiont Bacteroides fragilis or with the spore-forming Segmented Filamentous Bacteria (SFB) display deficits in serum 5-HT that are comparable to those seen in GF mice (FIG. 6A). Moreover, postnatal colonization (P42) with Bacteroides uniformis, altered Schaedler flora (ASF), an eight-microbe consortium known to correct gross intestinal pathology in GF mice (Dewhirst et al., Appl. Environ. Microbiol. 65:3287-3292 (1999)), or with cultured Bacteroides spp. from the SPF mouse microbiota, has no significant effect on the 5-HT deficiency seen in GF mice (FIGS. 6A and 6B). Interestingly, however, GF mice colonized at P42 with indigenous spore-forming microbes from the mouse SPF microbiota (Sp), known to be dominated by Clostridial species (see e.g., FIG. 14), exhibit complete restoration of serum and colon 5-HT to levels that match, or surpass, those observed in SPF mice (FIGS. 6A and 6B). Consistent with this, Sp colonization of GF mice increases 5-HT staining co-localized to CgA+ECs (FIGS. 4A-4D), elevates host colonic TPH1 expression (FIG. 6D) and decreases SLC6A4 expression (FIG. 6E) toward levels seen in SPF mice. Improvements in serum 5-HT are observed within 2 days after inoculation of GF mice with Sp (FIG. 5E), and do not correlate with amelioration of abnormal cecal weight (FIG. 5F). Importantly, Sp also elevates colonic 5-HT in Rag1 knockout mice (FIG. 5G), which lack adaptive immune cells, indicating that the effects of Sp on gut 5-HT are not dependent on Sp-mediated regulatory T cell induction. Notably, the 5-HT-promoting effects of Sp are recapitulated by colonization of GF mice with spore-forming microbes from the healthy human colonic microbiota (hSp) (FIGS. 7A-7D), demonstrating that the serotonergic function of this community is conserved across mice and humans.

To determine whether the effects of Sp on host peripheral 5-HT depend on colonic Tph activity, GF mice were colonized with Sp on P42 and then administered the Tph inhibitor parachlorophenylalanine (PCPA) intrarectally twice daily for 3 days prior to 5-HT assessments on P56 (Liu et al., J. Pharmacol. Exp. Ther. 325:47-55 (2008)). Intrarectal injection of PCPA sufficiently blocks the ability of Sp to elevate colon and serum 5-HT levels (FIGS. 6C and 5C), as well as Sp-mediated increases in 5-HT staining in ECs (FIGS. 4A-4D). Similar effects of PCPA treatment on blocking increases in colon 5-HT, serum 5-HT and 5-HT staining in colonic ECs are seen in GF mice colonized with hSp (FIGS. 7A-7D). Interestingly, inhibiting Tph activity with PCPA results in a compensatory increase in colonic TPH1 and decrease in SLC6A4 (FIGS. 6D and 5D) expression in Sp-colonized mice, supporting the notion that microbiota-dependent changes in 5-HT transporter levels occur as a secondary response to Tph modulation.

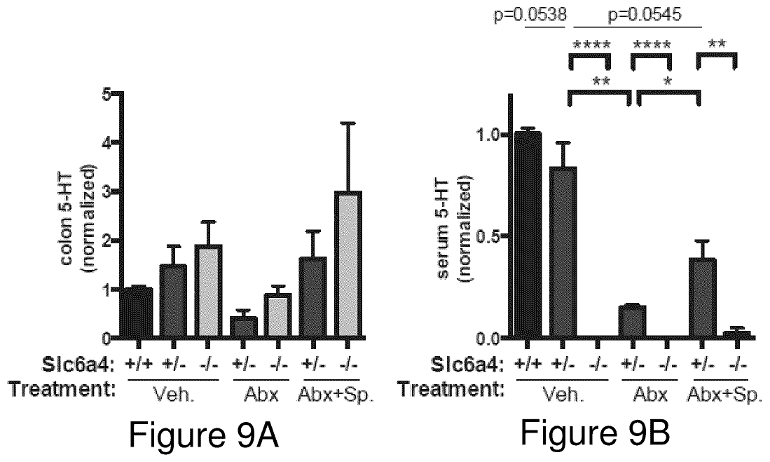

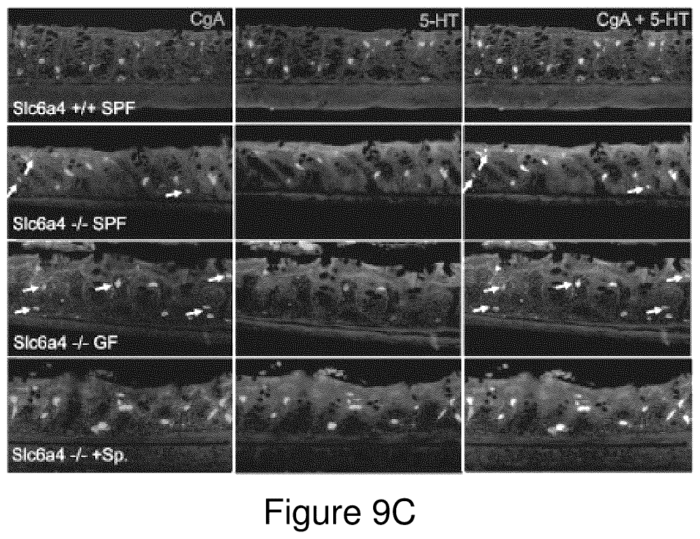

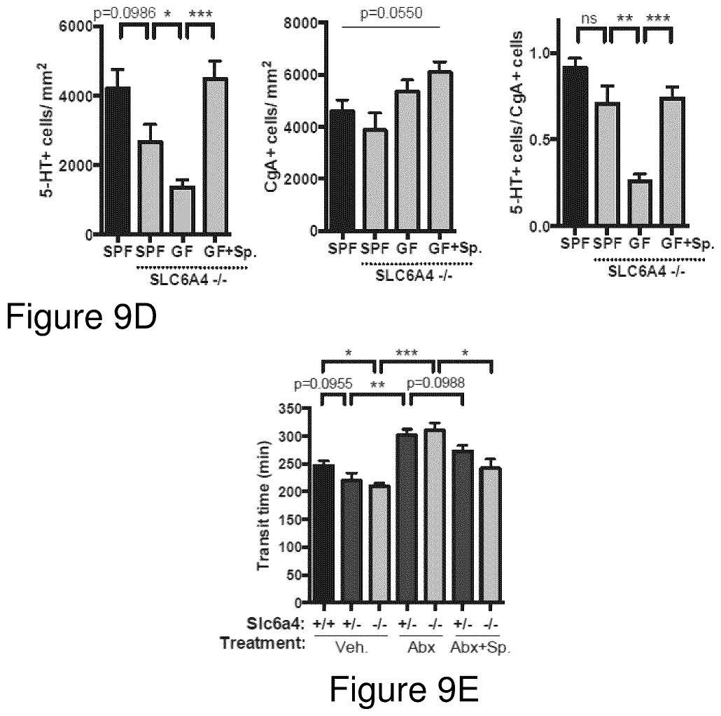

To further evaluate whether changes in SLC6A4 expression are necessary for microbiota-mediated alterations in peripheral 5-HT, the effects of microbiota manipulations were tested on colon and serum 5-HT in SLC6A4 heterozygous (+/−) and complete (−/−) knockout (KO) mice. Depleting the microbiota via P42-P56 antibiotic treatment (Reikvam et al., PLOS ONE 6: e17996 (2011)) of SPF SLC6A4+/− and −/−mice effectively decreases colonic 5-HT levels (FIGS. 9A and 9B), indicating that the microbiota is required for promoting gut 5-HT in Slc6a4-deficient mice. Colonizing antibiotic-treated SLC6A4+/− and −/−mice with Sp raises colon 5-HT to levels that match, or surpass, those seen in SPF SLC6A4+/− and −/−mice (FIG. 9A), demonstrating that Slc6a4 is not required for conferring the effects of Sp on gut 5-HT. Antibiotic-induced decreases and Sp-induced increases in colon 5-HT levels can be attributed to modulation of 5-HT content in colonic ECs from SLC6A4+/− and −/−mice (FIG. 9C). Similar effects of antibiotic treatment and Sp colonization are seen for serum 5-HT in SLC6A4+/− mice, whereas SLC6A4−/−mice exhibit low to undetectable levels of serum 5-HT, highlighting the dependence of platelets on Slc6a4-mediated 5-HT uptake (FIG. 9B).

Taken together, these data strongly support a role for indigenous spore-forming microbes in promoting host serotonin biosynthesis, for example by promoting Tph1-mediated 5-HT biosynthesis by colonic ECs, regulating both colon and serum levels of 5-HT.

### Example 3

### Microbiota-Mediated Regulation of Host Serotonin Modulates Gastrointestinal Motility

The present example demonstrates that segmented filamentous bacteria-mediated increases in colonic 5-HT biosynthesis are important for gut motility function.

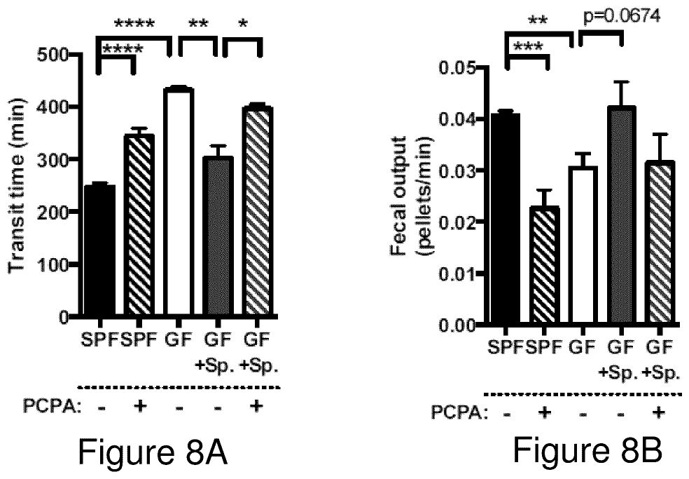

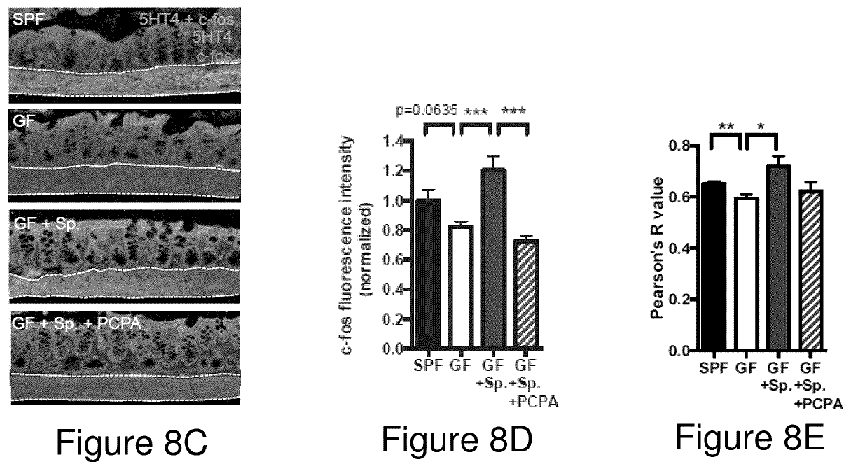

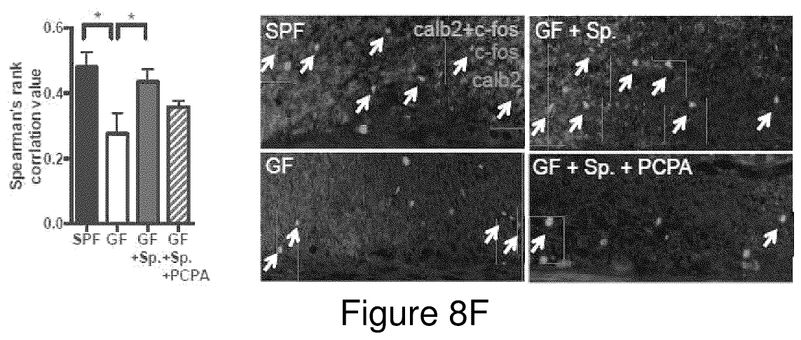

Intestinal 5-HT plays an important role in stimulating the enteric nervous system and GI function. To determine whether microbiota-dependent modulation of colonic 5-HT impacts GI motility, P42 GF mice were colonized with Sp and then tested for GI transit and colonic neuronal activation at P56. Sp colonization ameliorates GF-associated abnormalities in GI motility, significantly decreasing total transit time and increasing the rate of fecal output in a Tph-dependent manner (FIGS. 8A and 8B). Similar effects are seen in SLC6A4+/− and −/−mice, where Sp colonization of antibiotic-treated mice restores GI transit time toward levels seen in SPF SLC6A4+/− and −/−controls (FIG. 9E).

Consistent with deficits in GI motility, steady-state activation of 5-HT receptor subtype 4 (5HT4)-expressing neurons in the colonic submucosa and muscularis externa is decreased in GF mice compared to SPF controls, as measured by colocalized expression of 5HT4 with the immediate early gene, c-fos (FIGS. 8C, 8D and 8E). Colonization of GF mice with Sp increases 5HT4+c-fos+ staining to levels seen in SPF mice, and this effect is dependent on colonic Tph activity (FIGS. 8C, 8D and 8E), which aligns well with the understanding that Sp-induced elevations in colonic 5-HT promote GI motility by activation of 5HT4+ enteric neurons (Mawe and Hoffman, 2013). In addition, colonic activation of intrinsic afferent primary neurons (IPANs) of the myenteric plexus is decreased in GF mice and improved by colonization with Sp, as measured by colocalization of cfos and the IPAN marker, calretinin (Calb2) (FIG. 8F). Inhibiting Tph activity with PCPA decreases IPAN activation in Sp-colonized mice, suggesting that some IPAN responses to Sp depend on host 5-HT synthesis (FIG. 8F).

Altogether, these findings indicate that Sp-mediated increases in colonic 5-HT biosynthesis are important for gut sensorimotor function.

### Example 4

### Microbiota-Mediated Regulation of Host Serotonin Modulates Platelet Function

The present example demonstrates that elevations in colonic serotonin levels mediated by gut microbiota promote platelet activation and aggregation.

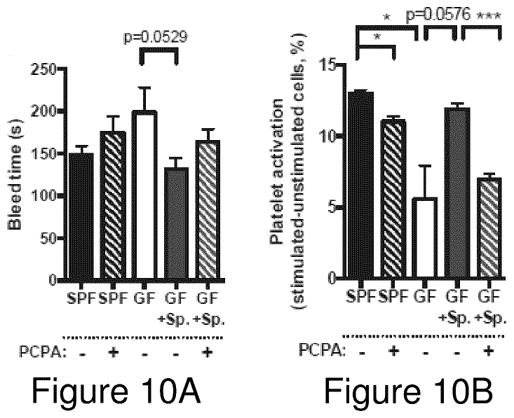

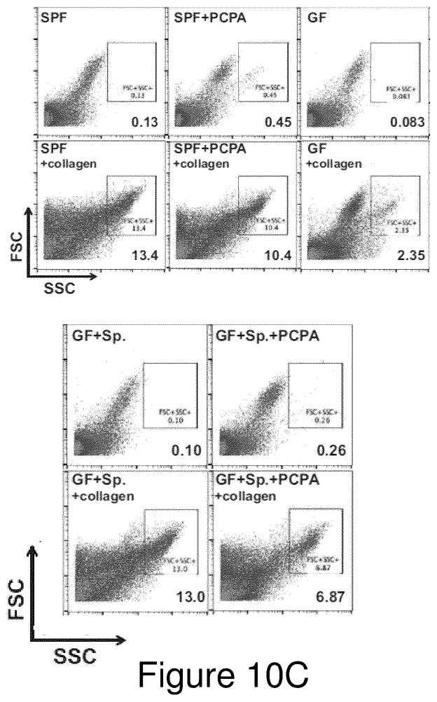

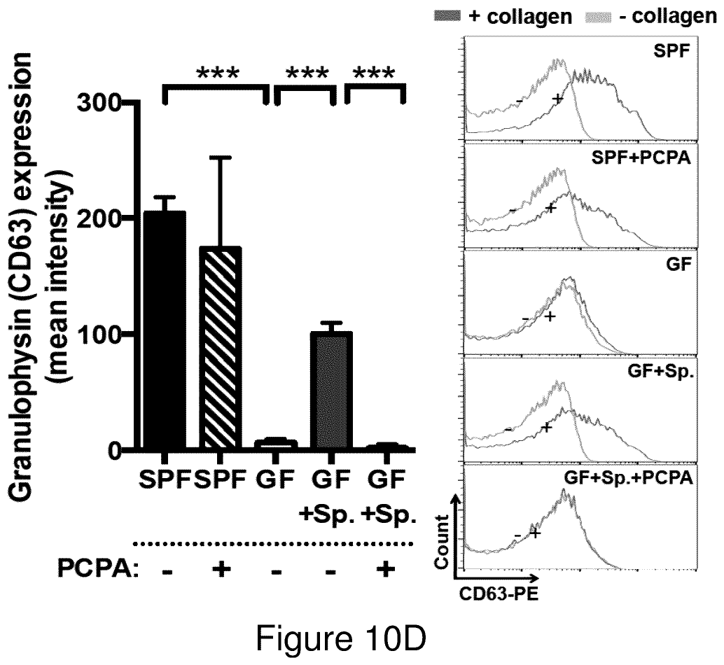

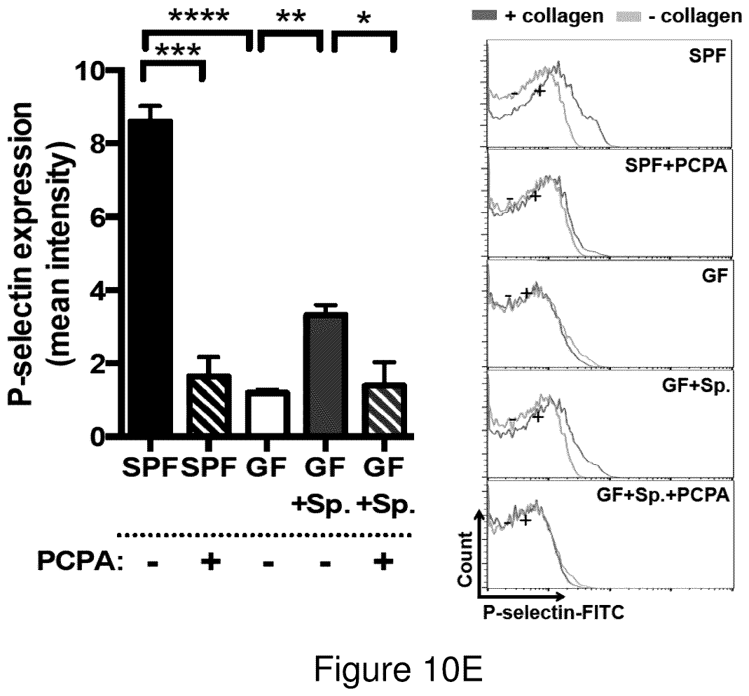

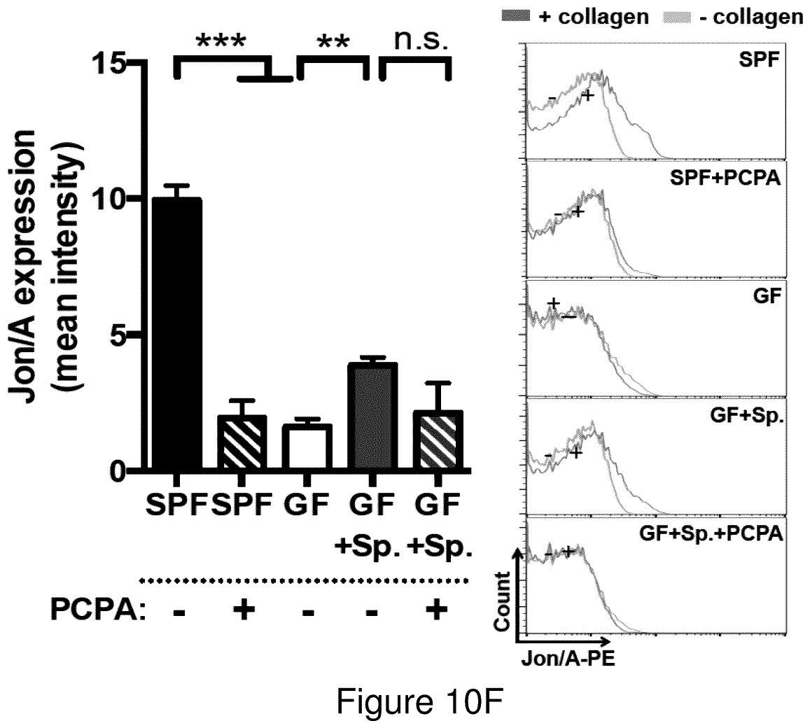

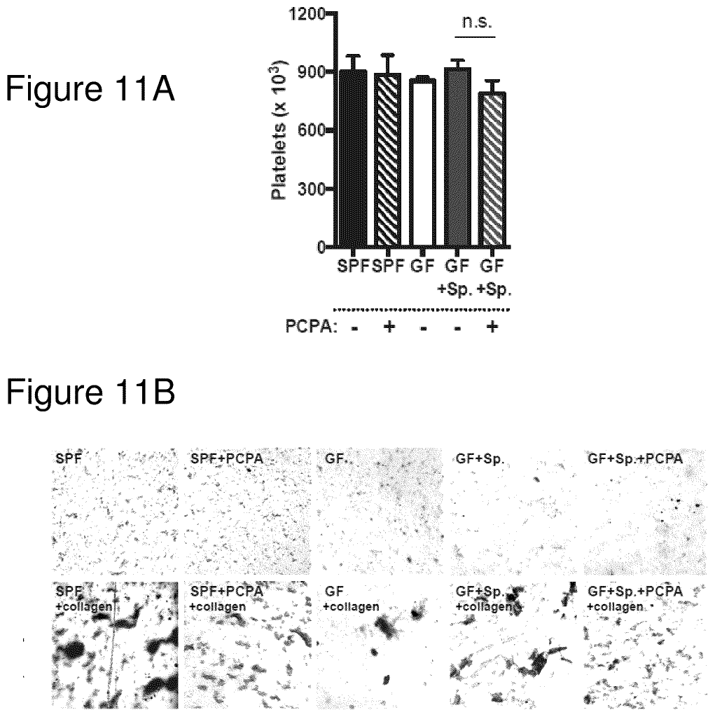

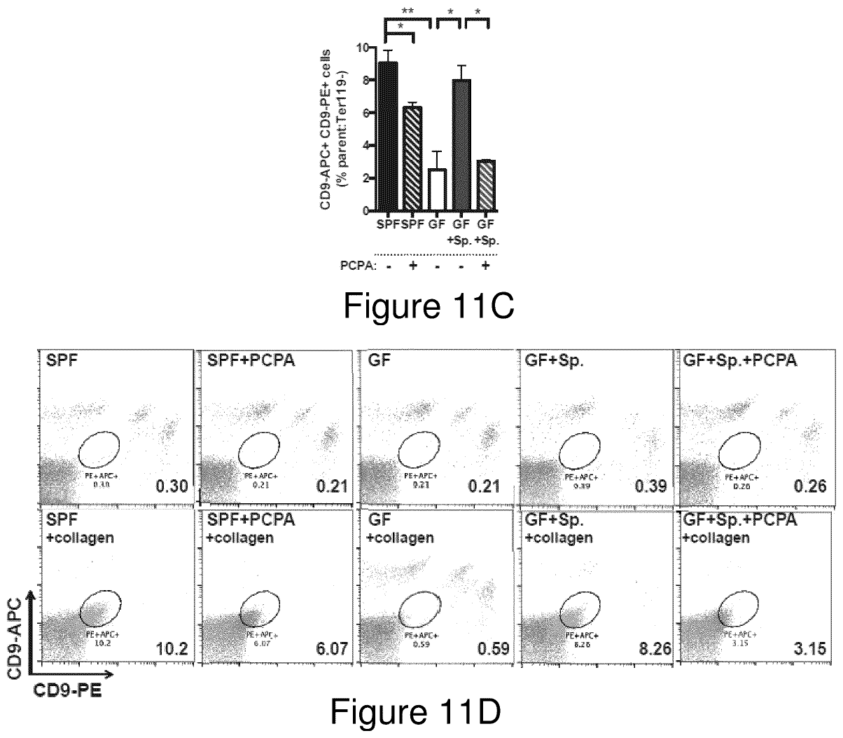

Platelets uptake gut-derived 5-HT and release it at sites of vessel injury to promote blood coagulation. To determine if microbiota-dependent modulation of colon (FIGS. 2A-2D and 6A-6D) and plasma (FIG. 6A) 5-HT impacts platelet function, P42 mice were colonized with Sp and then examined blood clotting, platelet activation and platelet aggregation at P56. In a tail bleed assay (Liu et al., World J. Exp. Med. 2:30-36 (2012)), GF mice exhibit trending increases in time to cessation of bleeding compared to SPF mice, suggesting impaired blood coagulation (FIG. 10A). Colonization of GF mice with Sp ameliorates abnormalities in bleeding time to levels seen in SPF controls, and this effect is attenuated by intrarectal administration of PCPA (FIG. 10A), indicating that Sp-mediated improvements in coagulation may be dependent on colonic Tph activity. Notably, the impact of acute colonic PCPA treatment on reducing 5-HT content and 5-HT-related functions in platelets may be tempered by the fact that mouse platelets have a lifespan of ˜4 days. There were no significant differences between treatment groups in total platelet counts (FIG. 11A).

In light of inherent limitations of the tail bleed assay, subsequent experiments were focused particularly on platelet activity. Platelets isolated from GF mice display decreased activation in response to in vitro type I fibrillar collagen stimulation, as measured by reduced surface expression of the activation markers granulophysin (CD63), P-selectin and JON/A (integrin α∥bβ3) (FIGS. 10D, 10E and 10F) (Ziu et al., J. Mol. Cell. Cardiol. 52:1112-1121 (2012)). Sp colonization of GF mice leads to partial restoration in the expression of platelet activation markers, and this effect depends on colonic Tph activity (FIGS. 10D, 10E and 10F). Moreover, platelets isolated from GF mice exhibit impaired aggregation in response to in vitro collagen stimulation, as measured by decreased levels of high granularity, high mass aggregates detected by both flow cytometry (side scatter (SSC)-high, forward scatter (FSC)-high events) (FIGS. 10B and 10C) and imaging (FIG. 11B). In another assay measuring platelet aggregation, platelets were separately labeled with either FITC or APC conjugated anti-CD9, and aggregation in response to stimulation was measured by detection of large FITC^{mid}, APC^{mid} events by flow cytometry (De Cuyper et al., 2013). Platelets from GF mice compared to SPF controls display decreased aggregation by this method (FIGS. 10C and 10E). Colonization of GF mice with Sp restores levels of platelet aggregation to those seen in SPF mice, increasing levels of high granularity (SSC), high mass (FSC) aggregates (FIGS. 10B and 10C), and increasing aggregation of fluorophore-labeled platelets as detected by flow cytometry (FIGS. 11C and 11D). These effects of Sp on correcting impaired platelet aggregation are attenuated by colonic PCPA injection (FIGS. 11C and 11D), indicating dependence on Tph activity.

Overall, these findings strongly suggest that Sp-mediated elevations in colonic 5-HT, and thus platelet 5-HT, promote platelet activation and aggregation relevant to hemostasis.

### Example 5

### Microbial Metabolites Mediate Effects of the Microbiota on Host Serotonin

Potential host-microbial interactions that regulate peripheral 5-HT were explored by surveying microbial influences on the fecal metabolome. This example identifies the microbial metabolites responsible for elevating serotonin levels through direct signaling to colonic enterochromaffin cells.

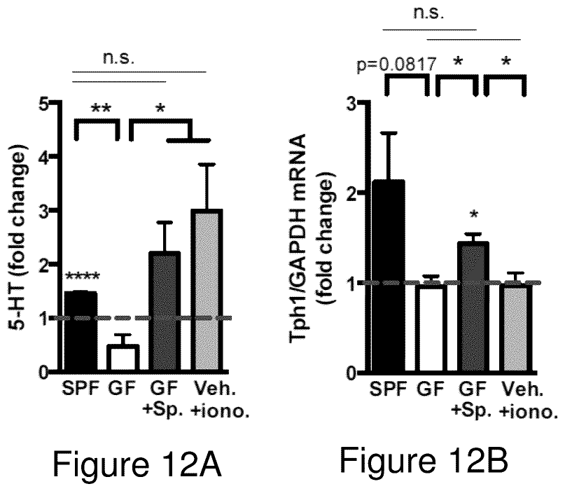

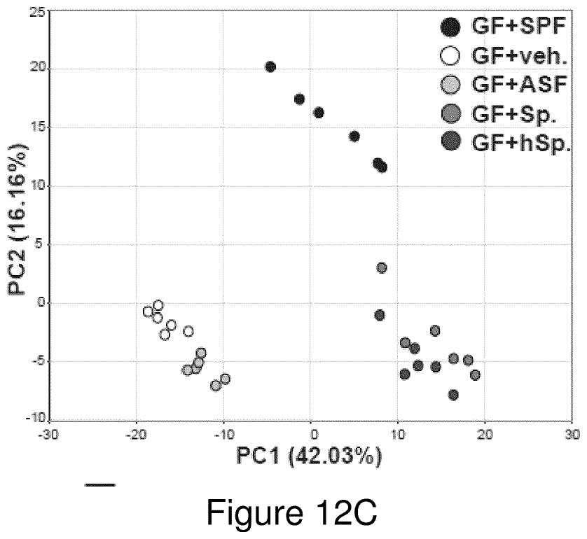

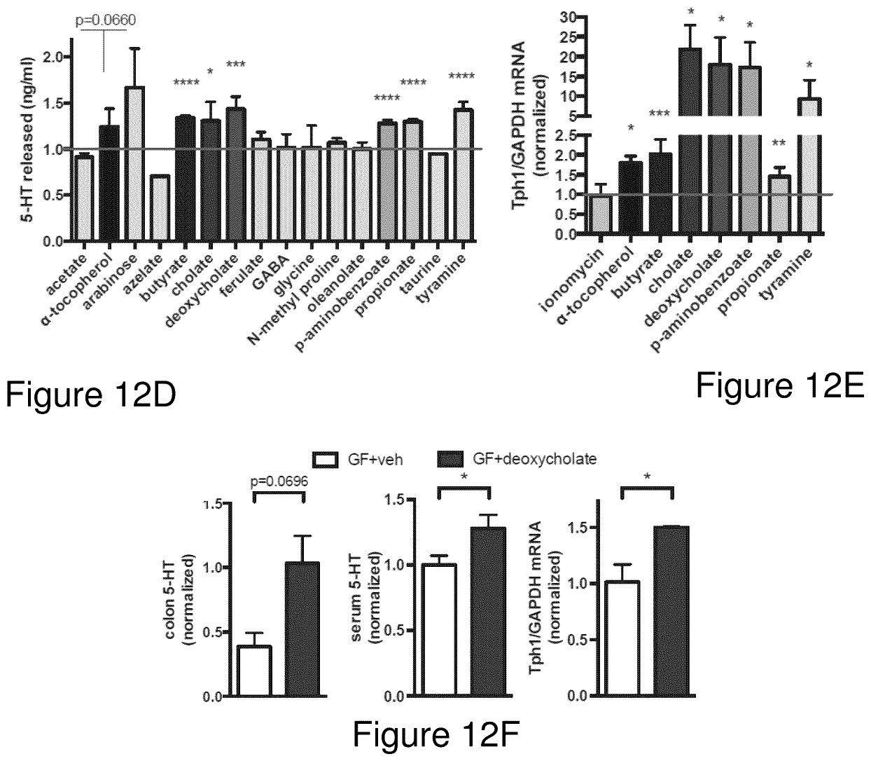

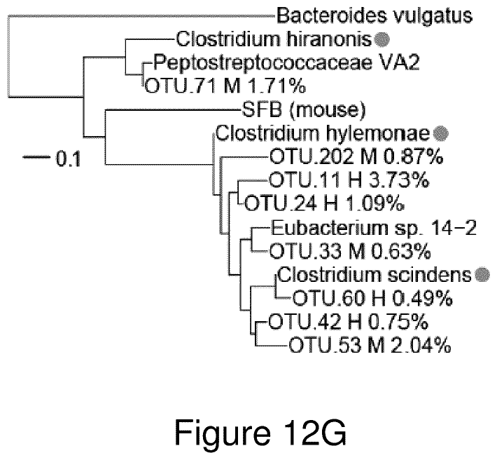

In light of the important role for Sp in regulating 5-HT-related intestinal and platelet function, the specific microbial factors responsible for conferring the serotonergic effects of Sp were identified. Based on our finding that Sp elevates 5-HT particularly in colonic ECs (FIGS. 4A-4D), it was hypothesized that Sp promotes levels of a soluble factor that signals directly to ECs to modulate TPH1 expression and 5-HT biosynthesis. To test this, filtrates of total colonic luminal contents from Sp-colonized mice and controls were prepared and their effects on levels of 5-HT in RIN14B chromaffin cell cultures were evaluated (Nozawa et al., 2009). Relative to vehicle-treated controls, there is no significant effect of filtered colonic luminal contents from GF mice on levels of 5-HT released or TPH1 expressed from RIN14B cells (FIGS. 12A and 12B). Filtered colonic luminal contents from SPF and Sp-colonized mice sufficiently induce 5-HT from RIN14B cells (FIG. 12A), to levels comparable to those elicited by the calcium ionophore, ionomycin, as a positive control (Nozawa et al., PNAS USA 106:3408-3413 (2009)). TPH1 expression is also elevated in cultured chromaffin cells exposed to SPF and Sp luminal filtrates, suggesting increased 5-HT synthesis. This is in contrast to ionomycin, which stimulates 5-HT release, but has no effect on TPH1 expression, from RIN14B cells. Importantly, these findings suggest that microbiota-mediated increases in gut 5-HT are conferred via direct signaling of a soluble, Sp-modulated factor to colonic ECs.

Gas chromatography/liquid chromatography with tandem mass spectrometry (GC/LC-MS)-based metabolomic profiling was utilized to identify candidate Sp-dependent, 5-HT-inducing molecules in feces from adult mice. 416 metabolites, spanning amino acid (92), peptide (111), carbohydrate (35), energy (6), lipid (90), nucleotide (29), xenobiotic (33) and cofactor and vitamin (20) super pathways were detected. Sp colonization of GF mice leads to statistically significant alterations in 75% of fecal metabolites detected, of which 76% are elevated and 24% are reduced, relative to vehicle-treated GF controls (Table 2). Similar alterations are seen with hSp colonization, which also alters 75% of metabolites detected (75% increased, 25% decreased), leading to co-clustering of Sp and hSp samples by principal components analysis (PCA) of fecal metabolomics data (FIG. 6C). ASF colonization has a mild effect, significantly modulating 50% of metabolites detected (66% increased, 36% decreased), as compared to controls (Table 2), and forming a distinct but proximal cluster to GF samples by PCA of metabolomics data (FIG. 12C). Postnatal conventionalization of GF mice with an SPF microbiota alters 66% of all metabolites detected (59% increased, 41% decreased), compared to vehicle-treated controls (Table 2), and produces substantial changes in the metabolome that are clearly distinguishable from the effects of Sp, hSp and ASF along PC2 (FIG. 12C). Notably, Sp, hSp and SPF colonization results in similar shifts along PC1, compared to vehicle and ASF-treated controls, suggesting common metabolic alterations among communities that similarly elevate peripheral 5-HT levels.

TABLE 2
Statistical Comparisons of Metabolomics Data by
Welch's Two-Sample t Test

| Group | Total | | Total | |
|---|---|---|---|---|
| com- | biochemical | Biochemicals | biochemical | Biochemicals |
| parisons | p ≤ 0.05 | (↑↓) | 0.05 < p < 0.10 | (↑↓) |
| GF | 274 | 111\|163 | 29 | 15\|14 |
| SPF | | | | |
| mSp | 215 | 187\|28 | 30 | 21\|9 |
| SPF | | | | |
| ASF | 258 | 122\|136 | 28 | 12\|16 |
| SPF | | | | |
| hSp | 229 | 186\|43 | 28 | 15\|13 |
| SPF | | | | |
| mSp | 312 | 238\|74 | 18 | 14\|4 |
| GF | | | | |
| ASF | 210 | 138\|72 | 24 | 17\|7 |
| GF | | | | |
| hSp | 311 | 232\|79 | 13 | 8\|5 |
| GF | | | | |
| ASF | 306 | 67\|239 | 29 | 14\|15 |
| mSp | | | | |
| hSp | 88 | 30\|58 | 24 | 7\|17 |
| mSp | | | | |
| hSp | 284 | 209\|75 | 24 | 14\|10 |
| ASF | | | | |

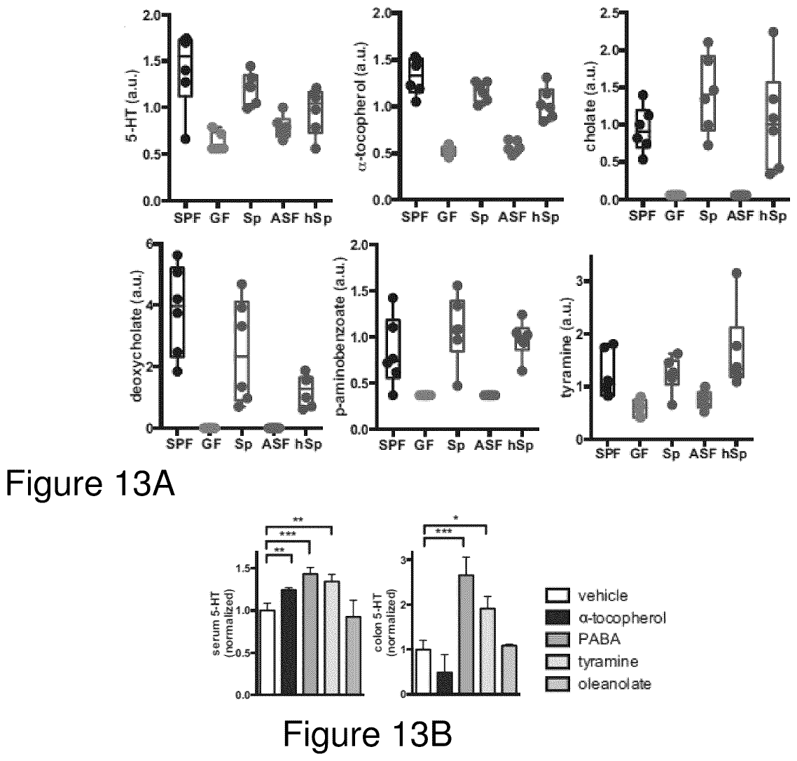

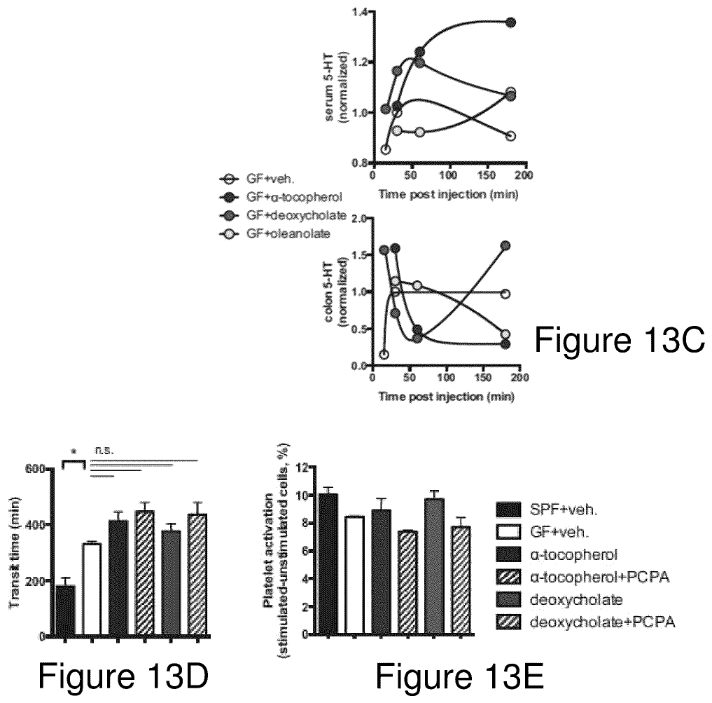

Without being bound by any particular theory, it was hypothesized that Sp, as well as hSp and SPF, colonization elevates levels of soluble metabolite(s) that increase 5-HT biosynthesis by colonic ECs, and that this effect is not seen by ASF colonization (FIG. 6A). Therefore, metabolites that were commonly upregulated in the Sp, hSp and SPF fecal metabolome, and comparatively low in ASF and GF samples were focused on. Metabolomics profiling confirms that fecal 5-HT follows this pattern, with increased levels of 5-HT in feces from Sp, hSp and SPF-colonized mice compared to ASF and GF controls. Simple linear regression reveals 83 metabolites that co-vary with 5-HT (r2≥0.25), 47 of which correlate positively and 36 of which correlate negatively with 5-HT levels (Table 3 and FIG. 13A). Several of the identified compounds are metabolically related, with enrichment of biochemicals relevant to i) plant fiber digestion, ii) pentose sugar metabolism, iii) bacterial bilirubin degradation, iv) bile acid metabolism, v) aromatic amino acid or plant phenolic compound digestion, vi) potential bacterial creatine metabolism, and vii) protein hydrolysis (Table 3).

TABLE 3
Correlation of Fecal Metabolites with Fecal Serotonin Levels

| Super | | | 5-HT | r |
|---|---|---|---|---|
| Pathway | Sub-pathway | Biochemical name | correlation | squared |
| Amino acid | Tryptophan | serotonin (5HT) | 1 | 1 |
| | metabolism | | | |
| Xenobiotics | Food | ferulate | 0.79 | 0.62 |
| | component/Plant | | | |
| Cofactors and | Tocopherol | alpha-tocopherol | 0.78 | 0.6 |
| vitamins | metabolism | | | |
| Xenobiotics | Food | oleanolate | 0.77 | 0.59 |
| | component/Plant | | | |
| Xenobiotics | Food | equol sulfate | 0.73 | 0.54 |
| | component/Plant | | | |
| Amino acid | Urea cycle; Arginine | N-methyl proline | 0.73 | 0.53 |
| | and Proline | | | |
| | Metabolism | | | |
| Carbohydrate | Pentose Metabolism | arabinose | 0.72 | 0.52 |
| Lipid | Fatty Acid, | azelate (nonanedioate) | 0.71 | 0.51 |
| | Dicarboxylate | | | |
| Cofactors and | Hemoglobin and | L-urobilin | 0.69 | 0.48 |
| vitamins | Porphyrin | | | |
| | Metabolism | | | |
| Carbohydrate | Pentose Metabolism | ribulose | 0.68 | 0.46 |
| Lipid | Secondary Bile Acid | deoxycholate | 0.67 | 0.45 |
| | Metabolism | | | |
| Lipid | Primary Bile Acid | cholate | 0.67 | 0.45 |
| | Metabolism | | | |
| Lipid | Primary Bile Acid | alpha-muricholate | 0.67 | 0.45 |
| | Metabolism | | | |
| Peptide | Dipeptide | isoleucyltyrosine | 0.67 | 0.44 |
| Lipid | Glycerolipid | glycerol | 0.66 | 0.44 |
| | Metabolism | | | |
| Cofactors and | Vitamin B6 | Pyridoxate (vitamin B6) | 0.66 | 0.44 |
| vitamins | Metabolism | | | |
| Amino acid | Phenylalanine and | 4-hydroxyphenylpyruvate | 0.65 | 0.43 |
| | Tyrosine | | | |
| | Metabolism | | | |
| Lipid | Long Chain Fatty | cis-vaccenate (18:1n7) | 0.65 | 0.43 |
| | Acid | | | |
| Lipid | Fatty Acid, | 13-methylmyristic acid | 0.65 | 0.42 |
| | Branched | | | |
| Amino acid | Methionine, | N-acetyltaurine | 0.65 | 0.42 |
| | Cysteine, SAM and | | | |
| | Taurine Metabolism | | | |
| Carbohydrate | Pentose Metabolism | xylose | 0.65 | 0.42 |
| Amino acid | Glycine, Serine and | sarcosine (N-Methylglycine) | 0.64 | 0.41 |
| | Threonine | | | |
| | Metabolism | | | |
| Lipid | Long Chain Fatty | oleate (18:1n9) | 0.64 | 0.4 |
| | Acid | | | |
| Carbohydrate | Aminosugar | N-acetylneuraminate | 0.63 | 0.39 |
| | Metabolism | | | |
| Lipid | Secondary Bile Acid | hyocholate | 0.6 | 0.36 |
| | Metabolism | | | |
| Carbohydrate | Fructose, Mannose | rhamnose | 0.6 | 0.35 |
| | and Galactose | | | |
| | Metabolism | | | |
| Lipid | Polyunsaturated | linolenate [alpha or gamma; | 0.59 | 0.34 |
| | Fatty Acid (n3 and | (18:3n3 or 6)] | | |
| | n6) | | | |
| Amino acid | Leucine, Isoleucine | 4-methyl-2-oxopentanoate | 0.58 | 0.33 |
| | and Valine | | | |
| | Metabolism | | | |
| Cofactors and | Hemoglobin and | coproporphyrin III | 0.57 | 0.33 |
| vitamins | Porphyrin | | | |
| | Metabolism | | | |
| Amino acid | Leucine, Isoleucine | 3-methyl-2-oxovalerate | 0.57 | 0.32 |
| | and Valine | | | |
| | Metabolism | | | |
| Carbohydrate | Aminosugar | N-acetylglucosamine | 0.56 | 0.31 |
| | Metabolism | | | |
| Lipid | Fatty Acid, | sebacate (decanedioate) | 0.53 | 0.29 |
| | Dicarboxylate | | | |
| Cofactors and | Hemoglobin and | D-urobilin | 0.53 | 0.28 |
| vitamins | Porphyrin | | | |
| | Metabolism | | | |
| Amino acid | Methionine, | taurine | 0.53 | 0.28 |
| | Cysteine, SAM and | | | |
| | Taurine Metabolism | | | |
| Peptide | Dipeptide | asparagylleucine | 0.52 | 0.28 |
| Xenobiotics | Food | equol | 0.52 | 0.27 |
| | component/Plant | | | |
| Peptide | Dipeptide | leucylglycine | 0.52 | 0.27 |
| Lipid | Long Chain Fatty | pentadecanoate (15:0) | 0.52 | 0.27 |
| | Acid | | | |
| Amino acid | Leucine, Isoleucine | 3-methyl-2-oxobutyrate | 0.51 | 0.26 |
| | and Valine | | | |
| | Metabolism | | | |
| Peptide | Dipeptide | glutamine-leucine | 0.51 | 0.26 |
| Nucleotide | Purine Metabolism, | guanine | 0.5 | 0.25 |
| | Guanine containing | | | |
| Amino acid | Phenylalanine and | 3-phenylpropionate | 0.5 | 0.25 |
| | Tyrosine | (hydrocinnamate) | | |
| | Metabolism | | | |
| Peptide | Dipeptide | phenylalanylglycine | 0.5 | 0.25 |
| Cofactors and | Tocopherol | delta-tocopherol | 0.5 | 0.25 |
| vitamins | metabolism | | | |
| Amino acid | Phenylalanine and | 2-(4-hydroxyphenyl)propionate | 0.5 | 0.25 |
| | Tyrosine | | | |
| | Metabolism | | | |
| Nucleotide | Purine Metabolism, | adenine | 0.5 | 0.25 |
| | Adenine containing | | | |
| Peptide | Dipeptide | serylleucine | 0.5 | 0.25 |
| Lipid | Short Chain Fatty | valerate | 0.5 | 0.25 |
| | Acid | | | |
| Carbohydrate | Aminosugar | glucosamine | −0.5 | 0.25 |
| | Metabolism | | | |
| Lipid | Lysolipid | 1- | −0.51 | 0.26 |
| | | palmitoylglycerophosphoinositol | | |
| Xenobiotics | Food | gluconate | −0.52 | 0.27 |
| | component/Plant | | | |
| Amino acid | Methionine, | cystine | −0.52 | 0.27 |
| | Cysteine, SAM and | | | |
| | Taurine Metabolism | | | |
| Carbohydrate | Fructose, Mannose | mannitol | −0.52 | 0.27 |
| | and Galactose | | | |
| | Metabolism | | | |
| Lipid | Polyunsaturated | urate | −0.52 | 0.27 |
| | Fatty Acid (n3 and | | | |
| | n6) | | | |
| Nucleotide | Purine Metabolism, | allantoin | −0.53 | 0.28 |
| | (Hypo)Xanthine/Inosine | | | |
| | containing | | | |
| Lipid | Inositol Metabolism | pinitol | −0.54 | 0.3 |
| Xenobiotics | Food | vanillate | −0.54 | 0.29 |
| | component/Plant | | | |
| Lipid | Inositol Metabolism | myo-inositol | −0.55 | 0.31 |
| Peptide | Gamma-glutamyl | gamma-glutamylthreonine | −0.55 | 0.31 |
| | Amino Acid | | | |
| Amino acid | Creatine Metabolism | creatine | −0.55 | 0.3 |
| Carbohydrate | Pentose Metabolism | arabitol | −0.55 | 0.3 |
| Lipid | Monoacylglycerol | 2-palmitoylglycerol (2- | −0.56 | 0.32 |
| | | monopalmitin) | | |
| Amino acid | Urea cycle; Arginine | pro-hydroxy-pro | −0.57 | 0.33 |
| | and Proline | | | |
| | Metabolism | | | |
| Amino acid | Alanine and | asparagine | −0.57 | 0.33 |
| | Aspartate | | | |
| | Metabolism | | | |
| Lipid | Long Chain Fatty | palmitate (16:0) | −0.57 | 0.32 |
| | Acid | | | |
| Cofactors and | Ascorbate and | arabonate | −0.58 | 0.34 |
| vitamins | Aldarate | | | |
| | Metabolism | | | |
| Carbohydrate | Pentose Metabolism | xylitol | −0.58 | 0.34 |
| Xenobiotics | Food | diaminopimelate | −0.58 | 0.34 |
| | component/Plant | | | |
| Amino acid | Urea cycle; Arginine | urea | −0.58 | 0.34 |
| | and Proline | | | |
| | Metabolism | | | |
| Amino acid | Phenylalanine and | 3-[3-(sulfooxy)phenyl]propanoic | −0.59 | 0.35 |
| | Tyrosine | acid | | |
| | Metabolism | | | |
| Carbohydrate | Fructose, Mannose | fructose | −0.6 | 0.36 |
| | and Galactose | | | |
| | Metabolism | | | |
| Lipid | Lysolipid | 1- | −0.6 | 0.36 |
| | | linoleoylglycerophosphoethanolamine | | |
| Lipid | Primary Bile Acid | tauro(alpha + beta)muricholate | −0.6 | 0.35 |
| | Metabolism | | | |
| Peptide | Gamma-glutamyl | gamma-glutamylvaline | −0.61 | 0.38 |
| | Amino Acid | | | |
| Lipid | Primary Bile Acid | taurocholate | −0.63 | 0.4 |
| | Metabolism | | | |
| Amino acid | Leucine, Isoleucine | 2-methylbutyrylcarnitine (C5) | −0.64 | 0.42 |
| | and Valine | | | |
| | Metabolism | | | |
| Lipid | Secondary Bile Acid | tauroursodeoxycholate | −0.64 | 0.41 |
| | Metabolism | | | |
| Cofactors and | Ascorbate and | threonate | −0.64 | 0.4 |
| vitamins | Aldarate | | | |
| | Metabolism | | | |
| Xenobiotics | Food | soyasaponin I | −0.65 | 0.42 |
| | component/Plant | | | |
| Xenobiotics | Food | soyasaponin II | −0.65 | 0.42 |
| | component/Plant | | | |
| Amino acid | Urea cycle; Arginine | dimethylarginine (SDMA + | −0.67 | 0.45 |
| | and Proline | ADMA) | | |
| | Metabolism | | | |
| Carbohydrate | Pentose Metabolism | xylonate | −0.7 | 0.5 |
| Amino acid | Methionine, | cysteine | −0.71 | 0.5 |
| | Cysteine, SAM and | | | |
| | Taurine Metabolism | | | |
| Peptide | Dipeptide | glycylglycine | −0.75 | 0.56 |

To determine whether specific metabolites mediate the effects of Sp on 5-HT, a subset of biochemicals that were commonly upregulated by Sp, hSp and SPF, and that positively correlated with 5-HT levels (Table 3 and FIG. 13A) were tested for their ability to induce 5-HT in vitro and in vivo. Also tested were the short chain fatty acids, acetate, butyrate and propionate, which were previously shown to be produced by Sp and to stimulate 5-HT release from ECs. Of the 16 metabolites examined, α-tocopherol, butyrate, cholate, deoxycholate, paminobenzoate (PABA), propionate and tyramine elevate 5-HT in RIN14B chromaffin cell cultures (FIG. 12D). Elevations in 5-HT correspond to substantial increases in TPH1 expression from RIN14B cells (FIG. 12E), suggesting that particular metabolites elevated by Sp enhance 5-HT biosynthesis by intestinal ECs. Further tested were select metabolites for sufficiency to induce 5-HT in vivo. Notably, raising luminal concentrations of deoxycholate in colons of GF mice to levels seen in SPF mice (Sayin et al., Cell metabolism 17:225-235 (2013)) sufficiently increases colon and serum 5-HT compared to vehicle-injected controls (FIGS. 12F and 13B). This restoration of peripheral 5-HT correlates with elevations in colonic TPH1 expression (FIG. 12F), demonstrating that colonic signaling of deoxycholate significantly impacts host 5-HT metabolism. Increases in colon and serum 5-HT are also seen with injection of α-tocopherol, PABA and tyramine into colons of GF mice (FIGS. 13B and 13C). Consistent with in vitro RIN14B culture data, oleanolate has no statistically significant effect on elevating colon or serum 5-HT in GF mice (FIGS. 13B and 13C). Importantly, the effects of a single rectal injection of deoxycholate or α-tocopherol on raising colon 5-HT levels in GF mice are weak and transient, peaking within 1 hour of injection (FIG. 13C). Consistent with this, there is no significant effect of acute colonic metabolite injection on GI transit time (FIG. 13D), and there is only a trending improvement on platelet activation (FIG. 13E). Our finding that Sp colonization leads to lasting increases in colon and blood 5-HT levels (FIGS. 12A-12G), and long-term changes in the fecal metabolome (see e.g., FIG. 12C and Tables 3), suggests that Sp colonization results in persistent elevations of 5-HT-modulating luminal metabolites. Future studies on whether chronic, colon-restricted increases in Sp-regulated metabolites sufficiently correct GI motility and platelet function in GF mice, and whether this occurs in a 5-HT-dependent manner, are warranted. In addition, these studies surprisingly demonstrate that select concentrations of Sp-associated metabolites sufficiently promote 5-HT in vitro and in vivo, but whether the metabolites are necessary for mediating the serotonergic effects of Sp is unclear. Interestingly, several members of the Sp microbiota are phylogenetically related to species with the genetic capacity to modulate deoxycholate, α-tocopherol and tyramine (FIG. 14).

Overall, these data reveal that indigenous spore-forming microbes promote 5-HT biosynthesis, for example 5-HT biosynthesis from colonic ECs, modulating 5-HT concentrations in the host (e.g., in host colon and blood). Furthermore, select microbial metabolites that confer the serotonergic effects of indigenous spore-forming microbes were identified, likely by signaling directly to colonic ECs to promote Tph1 expression and 5-HT biosynthesis.

Examples 1-5 described above demonstrate that the gut microbiota plays a key role in promoting 5-HT levels in subjects, such as colon and blood 5-HT level, largely by elevating synthesis by host colonic ECs. This host-microbiota interaction contributes to a growing appreciation that the microbiota regulates many aspects of GI physiology such as intestinal barrier integrity, stem cell differentiation and enteroendocrine L cell function, by signaling to host cells.

### Example 6

### Treatment of Irritable Bowel Syndrome

This example illustrates the treatment of a patient suffering from Irritable Bowel Syndrome (IBS).

A patient is identified as being suffering from IBS. The blood level of one or more of deoxycholate, α-tocopherol, tyramine, and p-aminobenzoate in the patient is determined. A probiotic composition containing Lactobacillales, Proteobacteria, Clostridia, or a mixture thereof is administered to the patient via oral administration. The administration of the probiotic composition is expected to alter the blood level of one or more of deoxycholate, α-tocopherol, tyramine, and p-aminobenzoate, and composition of gut microbiota in the patient. It is also expected that the probiotic administration will relieve one or more symptoms of IBS in the patient.

### Example 7

### Treatment of Abnormal GI Motility

This example illustrates the treatment of a patient suffering from abnormal GI motility.

A patient is identified as being suffering from abnormal GI motility. The blood level of one or more of deoxycholate, α-tocopherol, tyramine, and p-aminobenzoate in the patient is determined. A probiotic composition containing Clostridia Cluster IV bacteria, Clostridia Cluster XIVa bacteria, or a mixture thereof is administered to the patient via oral administration. The administration of the probiotic composition is expected to alter the blood level of one or more of deoxycholate, α-tocopherol, tyramine, and p-aminobenzoate, and composition of gut microbiota in the patient. It is also expected that the probiotic administration will restore GI motility to normal in the patient.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for modulating the level of serotonin in a subject, comprising:
adjusting the composition of gut microbiota in a subject, and thereby changing the level of serotonin in the subject.

2. The method of claim 1, further comprising determining the level of serotonin in the subject before the composition of gut microbiota in the subject is adjusted, after the composition of gut microbiota in the subject is adjusted, or both.

3. The method of claim 1, wherein the subject suffers from or is at a risk of developing a serotonin-related disease.

4. The method of claim 3, wherein the serotonin-related disease is irritable bowel syndrome, inflammatory bowel disease, cardiovascular disease, osteoporosis, abnormal gastrointestinal motility, abnormal platelet aggregation, abnormal platelet activation, abnormal immune response, depression, anxiety, or a combination thereof.

5. The method of claim 1, wherein the subject suffers from an abnormality in enteric motor and secretory reflexes, an abnormality in platelet aggregation, an abnormality in immune responses, an abnormality in bone development, an abnormality in cardiac function, an abnormality in gastrointestinal motility, an abnormality in hemostasis, an abnormality in mood, an abnormality in cognition, an abnormality in osteoblast differentiation, an abnormality in hepatic regeneration, an abnormality in erythropoiesis, an abnormality in intestinal immunity, an abnormality in neurodevelopment, or any combination thereof.

6. The method of claim 1, wherein changing the level of serotonin in the subject comprises changing one or more of the gut level, the colonic level, the peripheral level, the serum level, the plasma level, and the fecal level of serotonin in the subject.

7. The method of claim 1, wherein adjusting the composition of gut microbiota of the subject comprises fecal transplantation, microbiota conventionalization, microbial colonization, reconstitution of gut microbiota, probiotic treatment, antibiotic treatment, or a combination thereof.

8. The method of claim 1, wherein adjusting the composition of gut microbiota of the subject comprises administering to the subject a composition comprising one or more types of spore-forming bacteria.

9. The method of claim 8, wherein the one or more types of spore-forming bacteria comprise Lactobacillales, Proteobacteria, Clostridia, or a mixture thereof.

10. The method of claim 8, wherein the one or more types of spore-forming bacteria comprise Clostridia Cluster IV bacteria, Clostridia Cluster XIVa bacteria, or both.

11. The method of claim 8, wherein the composition comprising one or more types of spore-forming bacteria comprises spore-forming microbes from a human intestine.

12. The method of claim 8, wherein at least 50% of the bacteria in the composition comprising one or more types of spore-forming bacteria are Clostridial species.

13. The method of claim 8, wherein the composition comprising one or more types of spore-forming bacteria is a probiotic composition, a neutraceutical, a pharmaceutical composition, or a mixture thereof.

14. A method for modulating serotonin biosynthesis in a subject, comprising:
determining the level of one or more serotonin-related metabolites in a subject; and
adjusting the level of at least one of the one or more serotonin-related metabolites in the subject, and thereby modulating serotonin biosynthesis in the subject.

15. The method of claim 14, wherein adjusting the level of at least one of the one or more serotonin-related metabolites comprises adjusting the composition of gut microbiota in the subject.

16. The method of claim 15, wherein adjusting the composition of gut microbiota of the subject comprises administering the subject a composition comprising one or more types of spore-forming bacteria.

17. (canceled)

18. The method of claim 14, wherein adjusting the level of the serotonin-related metabolite in the subject comprises administering the serotonin-related metabolite to the subject, activating an enzyme involved in the in vivo synthesis of the serotonin-related metabolite, administering a substrate or an intermediate in the in vivo synthesis of the serotonin-related metabolite, or a combination thereof.

19. The method of claim 14, wherein adjusting the level of the serotonin-related metabolite in the subject comprises inhibiting an enzyme involved in the in vivo synthesis of the serotonin-related metabolite, administering to the subject an antibody against the serotonin-related metabolite, administering to the subject an antibody against an intermediate for the in vivo synthesis of the serotonin-related metabolite, administering to the subject an antibody against a substrate for the in vivo synthesis of the serotonin-related metabolite, or a combination thereof.

20. The method of claim 18, wherein the serotonin-related metabolite is deoxycholate, α-tocopherol, paminobenzoate, or tyramine.

21. A method for treating a disorder caused by serotonin deficiency, comprising:
adjusting the composition of gut microbiota in a subject suffering from a disorder caused by serotonin deficiency; and
increasing the colonic or blood level of serotonin in the subject.

22-27. (canceled)
